(19) United States
(12) Patent Application Publication
(10) Pub. No.: US 2012/0270774 A1
(43) Pub. Date: Oct. 25, 2012

(54) MACROCYCLIC COMPOUNDS, COMPOSITIONS COMPRISING THEM AND METHODS FOR PREVENTING OR TREATING HIV INFECTION

(75) Inventors: Chaim Gilon, Jerusalem (IL); Amnon Hoffman, Jerusalem (IL); Moshe Kotler, Mevasseret Zion (IL); Mattan Hurevich, Jerusalem (IL); Salim Joubran, Lod (IL); Avi Swed, Holon (IL)

(73) Assignee: Yissum Research Development Company of the Hebrew University of Jerusalem LTd, Jerusalem (IL)

(21) Appl. No.: 13/392,846

(22) PCT Filed: Aug. 29, 2010
(86) PCT No.: PCT/IL10/00708

§ 371 Date: Jun. 27, 2012

Related U.S. Application Data
(60) Provisional application No. 61/237,779, filed on Aug. 28, 2009.

(51) Int. Cl. A61K 38/12; A61P 31/18; A61P 31/12; C07K 5/12

(52) U.S. Cl. 514/3.8; 540/460; 514/21.1; 514/3.7

(57) ABSTRACT

The present invention relates to backbone cyclized CD-4 mimetics and to compositions and methods comprising them for preventing and treating viral infection. In particular, the present invention relates to orally bio-available compounds and formulations for prevention and treatment of human HIV-1 infection.

### FIELD OF THE INVENTION

The present invention relates to CD4 mimetic compounds, to compositions comprising them and to methods for using them in prevention and treatment of HIV infection, particularly HIV-1 infection.

### BACKGROUND OF THE INVENTION

The Human Immunodeficiency Virus (HIV) retrovirus is responsible for AIDS (acquired immunodeficiency syndrome), an incurable disease in which the body's immune system breaks down leaving it vulnerable to opportunistic infections, such as pneumonia, and certain cancers. AIDS is a major global health problem. Since the beginning of the epidemic, almost 60 million people have been infected with HIV and 25 million people have died of HIV-related causes. AIDS has replaced malaria and tuberculosis as the world's deadliest infectious disease, and is the fourth leading cause of death in the world. In 2008, some 33.4 million people were living with HIV and round 430 000 children were born with HIV, bringing to 2.1 million the total number of children under 15 living with HIV.

AIDS remains a major disease that is elusive of a cure after almost two decades of intense search for an effective treatment. Currently available HIV drugs include reverse transcriptase (RT) and protease inhibitors (PR). Although drug combination regimens has results in significant decline of AIDS related death in the developed world, 78% of HIV patients with measurable viral loads carry virus that is resistant to one or more drugs. Furthermore, many of the newly diagnosed HIV patients are infected with resistant viruses. Compounds with novel anti-HIV targets are therefore required. Agents that interfere with HIV entry into the cell represent one class of inhibitors suggested for treating HIV infections (D'Souza et al., 2000, JAMA 284, 215-222).

The major problem in developing an efficient drug against AIDS is the virus tendency to mutate. Since HIV is an organism with relatively primitive control mechanisms, this virus, like many other retroviruses, tends to have a high mutation rate. This high mutation rate causes frequent generation of various viral types, so when exposed to the drugs in use, shortly a resistant type is formed. Thus, one of the challenges facing researchers today is developing an irresistible anti HIV drug. A drug of this sort should target a conserved viral site. However, any mutation in the viral site could lead the drug to becoming non-functional.

HIV envelope consists of an exterior glycoprotein gp120 and a transmembrane domain gp41. The HIV entry process involves the initial contact between the gp120 and the host cell CD4 receptor (Doms, R. W. and Moore, J. P., 2000, J. Cell. Biol. 151, F9-F14.). Subsequent conformational changes facilitate the binding of gp120 to the co-receptor CCR5 or CXCR4 and the insertion of the fusion peptide into the host membrane, finally resulting in fusion of the virus and cell membranes.

Agents targeting the HIV entry process are categorized into three groups based on the mode of action: (I) GP120/CD4 binding inhibitors; (II) Co-receptor inhibitors and (III) GP41 fusion peptide inhibitors.

### CD4 and CD4 Mimetics

CD4 is a mostly extra-cellular co-receptor embedded in the T cell membrane by a trans-membranal domain, followed by a short intra-cellular domain. This protein is very important in proper function of the immune system, mainly in the binding of CD4+ T cells to antigen presenting cells.

The truncated form of CD4 (sCD4) competes with the cell associated CD4 receptor for gp120 binding, therefore the protein exhibited potent antiviral activity against HIV-1. Yet, initial efforts to develop soluble CD4 as an anti-HIV agent failed due to its short serum half-life and its lack of activity against clinical HIV-1 isolates (Daar et al., 1990, Proc. Natl. Acad. Sci. USA 87, 6574-6578).

The recombinant CD4-IgG2 fusion proteins PRO542 produced by Progenic Pharmaceuticals demonstrated improved half-life in blood and achieved inhibitory activity over a broad range of HIV subtypes (Jacobson et al., 2000, J. Infect. Dis. 182, 326-329, Jacobson et al., 2004, Antimicrobial Agents and Chemotherapy, 48, 423-429), and this compound has entered phase II trial in an IV formulation. Other CD4 peptide mimics have been shown to have affinities to gp120 too weak to produce significant anti-HIV activity.

The crystal structure of a ternary complex composed of gp120 with the V1V2V3 loop-deleted the D1D2 domain CD4 and the Fab fragment of a CD41 monoclonal antibody has been reported (Furuta et al., 1998, Nat. Struct. Biol. 5, 276-279).

The most important residue in the CD4-gp120 binding site is CD4's Phe43. This residue is situated on a type II′ β-turn and its phenyl ring enters a hydrophobic pocket in gp120. This residue is responsible for 23% of the binding interactions between the two proteins, either by hydrophobic interactions of its phenyl ring or by both hydrophobic and hydrophilic interactions of its backbone atoms. It interacts with many gp120 residues: Glu370, Ile371, Asn425, Met426, Trp427, Gly473 and Asp368. Only the interaction with Ile371 is a classical hydrophobic one. There is also an aromatic stacking interaction of its phenyl ring with the carboxylate group of Glu370. Other interactions involve backbone atoms only. The second important residue is Arg59 of CD4. This residue forms a hydrogen bond with Asp368 of gp120. Residues Lys46, Lys35 and Lys29 are less important. Residues Asp368, Glu370 and Trp427, as well as the residues forming the hydrophobic pocket of gp120, were found to be conserved amongst various HIV strains. This shows their high importance in activity. A few point mutations were found to increase the binding affinity of the two proteins. Replacing Arg59 with a Lys residue tripled binding affinity, while replacing Gln40 or Asp63 by Ala residue doubles it.

Zhang et al. (Nature Biotechnology 1997, 15, 150-154) discloses constrained aromatically modified analogs of the secondary structure of the first domain of CD4 (synthetic CDRs of the D21 domain of CD4), which inhibit virus binding of HIV-1 to CD4 and virus replication in T lymphocytes.

PCT patent application WO 99/24065 discloses some theoretical inhibitors based on the crystal structure of gp120, which could interfere with gp120/CD4 interaction, through binding with the amino acid residues located in the D1D2-CD4 binding region of gp120. The possible inhibitors claimed are purely theoretical at this time. The inventors of WO 99/24065 have so far failed to produce any, of the inhibitors disclosed in the PCT publication possessing the specified chemical characteristics and anti-HIV activity.

US Patent Application 20040162298 describes a method of inhibiting HIV infection in a mammal by administering a small molecule compound having a molecular weight of less than about 1,000 dalton, wherein the compound interacts with HIV-gp120 and cause conformational change in the gp120 thereby preventing interaction between said gp120 and leukocyte CD4. The invention is exemplified by use of three small molecule compounds BMS-216, BMS-853 and BMS-806 disclosed in U.S. Pat. Nos. 6,469,006 and 6,476,034. The patents disclose that the compounds can be orally administered.

WO 2006/137075 to some of the inventors of the present application, provides backbone-cyclized molecules that mimic the gp120-binding site of the human CD4 protein and inhibit the HIV binding to the cells.

There is an unmet need for effective, metabolically stable and tissue permeable molecules for prevention and treatment of HIV infection. In particular, there is an unmet need for orally bio-available compositions and formulations against HIV-1 infection.

### SUMMARY OF THE INVENTION

The present invention provides improved compounds that mimic the gp120-binding site of the human CD4 protein. The compounds of the present invention are macrocyclic molecules characterized by having improved in-vivo stability, tissue permeability and oral bioavailability. The present invention further provides pharmaceutical compositions, formulations and methods for administration, particularly oral administration of CD4 mimetics.

The present invention provides, according to one aspect, analogs and derivatives of the macrocyclic compound of Formula I:

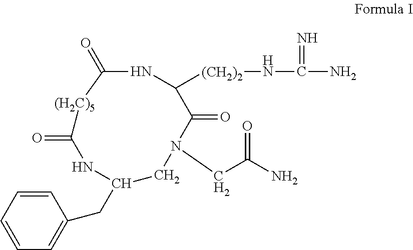

According to some embodiments, the macrocyclic derivative is according to Formula II:

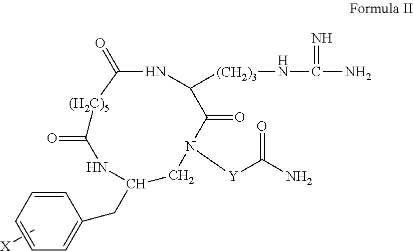

wherein X is hydrogen or is an electron withdrawing group, and Y is selected from the group consisting of: (CH₂)ₙ wherein n is 1-5; and CHR wherein R is an amino acid side chain.

According to some embodiments the electron withdrawing group is a halogen or a hydroxyl.

According to some embodiments X is a halogen group selected from the group consisting of: fluoride (F), chloride (Cl), bromide (Br) and iodide (I).

According to some specific embodiments, the macrocyclic compound is selected from the group consisting of:

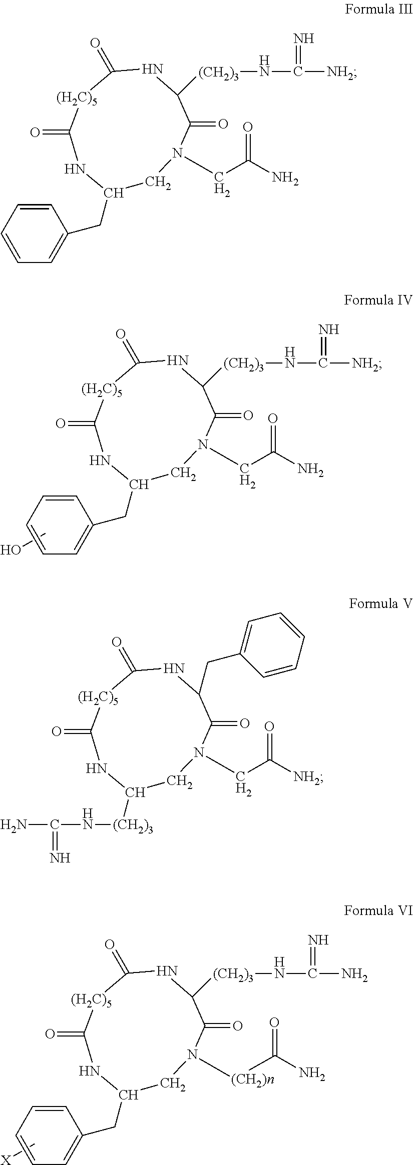

wherein X is hydrogen or is an electron withdrawing group; and n is 2-5;

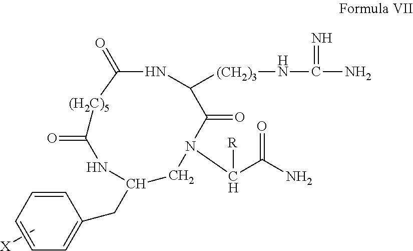

wherein X is a hydrogen or is an electron withdrawing group; and R is an amino acid side chain.

According to some embodiments a compound according to Formula VII is provided wherein R is other than Hydrogen.

According to some specific embodiments the present invention provides Phe derivatives of the compound of Formula III. According to certain embodiments, the Phe derivatives are Phe-halide derivatives. According to some specific embodiments the Phe-halide derivative is selected from the group consisting of: Phe-fluoride, Phe-chloride, Phe-bromide and Phe-iodide as presented in general formula VIII:

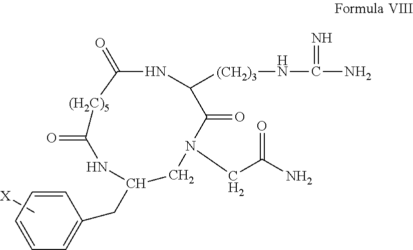

wherein X is selected from the group consisting of: fluoride (F), chloride (Cl), bromide (Br) and iodide (I).

According to yet other embodiments, urea-bond containing macrocyclic compounds are provided. According to some specific embodiments the urea-bond containing macrocyclic molecules are selected from compounds of Formula IX and Formula X, and analogs and derivatives of these molecules:

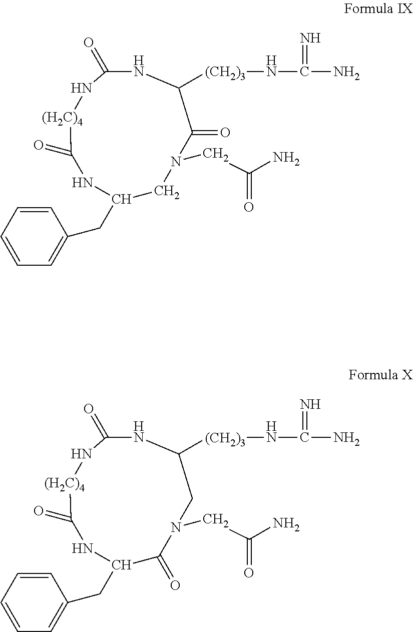

These molecules showed high permeability in Caco-2 model indication their potential bio- and oral-availability.

According to yet other embodiments, the macrocyclic CD4 mimetic is according to a formula selected from the group consisting of: Formula IV to Formula X and analogs and derivatives thereof.

According to additional embodiments the macrocyclic CD4 mimetic is according to VI or Formula VI and analogs and derivatives thereof.

According to a specific embodiment the macrocyclic CD4 mimetic is according to Formula II.

According to other specific embodiments the macrocyclic CD4 mimetic is according to Formula III.

The present invention provides, according to another aspect a pharmaceutical composition comprising as an active ingredient, at least one CD4 mimetic, particularly a backbone-macrocyclic molecule that mimics the non-contiguous active site of the human CD4 protein, and a pharmaceutically acceptable carrier or diluent.

According to some embodiments the pharmaceutical composition comprises at least one macrocyclic compound according to any one of Formulae IV-X.

According to other embodiments the pharmaceutical composition comprises a macrocyclic compound according to Formula II.

According to yet other embodiments the pharmaceutical composition comprises a macrocyclic compound according to Formula III.

Pharmaceutical compositions for use in accordance with the present invention may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

According to some embodiments an orally bioavailable composition of a compound according to Formula II or Formula III is provided. It is unexpectedly demonstrated that this compound, although having very low permeability coefficient value in the Caco-2 model, is oral bio-available as demonstrated in ex-vivo model.

According to yet additional embodiments, a pharmaceutical composition comprising at least one CD4 mimetic according to the invention and at least one additional retroviral inhibitor is provided.

According to some embodiments, the additional retroviral inhibitor is selected from the group consisting of: 3′-azido-3′-deoxythymidine (AZT), didanosine (dideoxy inosine; ddI), zalcitabine (dideoxycytidine; ddC), tenofovir (Viread®), or lamivudine (3′-thia-2′-3′-dideoxycytidine; 3TC). Anti-retroviral compounds also include non-nucleoside reverse transcriptase inhibitors such as suramine, foscarnet-sodium, nevirapine, sustiva and tacrine; TIBO type compounds; α-APA type compounds; TAT inhibitors (e.g., RO-5-3335); protease inhibitors (e.g., indinavir, ritonavir, saquinovir); NMDA receptor inhibitors (e.g., pentamidine); α-glycosidase inhibitors (e.g., castanospermine); Rnase H inhibitors (e.g., dextran); and immunomodulating agents (e.g., levamisole, thymopentin).

According to some embodiments the additional retroviral inhibitor is protease inhibitor.

According to specific embodiments the additional retroviral inhibitor is a CYP-3A4 inhibitor.

According to some specific embodiments the CYP-3A4 inhibitor is ritonavir.

According to some embodiments the molecule's scaffold confers permeability of the molecule. According to other embodiments the molecule comprises a permeability enhancing moiety. According to yet other embodiments, the permeability enhancing moiety is a peptide.

Any moiety known in the art to actively or passively facilitate or enhance permeability of the compound into cells may be used for conjugation with the molecules of the present invention. Non-limitative examples include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides.

According to another aspect, the present invention provides a formulation for oral administration comprising at least one backbone macrocyclic molecule which mimics the gp120 binding site of CD4. According to some embodiments the backbone macrocyclic molecule is according to Formula II or an analog or derivative thereof.

According to some embodiments the formulation for oral administration further comprises an exipient, carrier or diluent suitable for oral administration. Suitable pharmaceutically acceptable excipients for use in this invention include those known to a person ordinarily skilled in the art such as diluents, fillers, binders, disintegrants and lubricants. Diluents may include but not limited to lactose, microcrystalline cellulose, dibasic calcium phosphate, mannitol, cellulose and the like. Binders may include but not limited to starches, alginates, gums, celluloses, vinyl polymers, sugars and the like. Lubricants may include but not limited to stearates such as magnesium stearate, talc, colloidal silicon dioxide and the like.

The present invention provides, according to another aspect, a method for prevention, alleviation or treatment of a viral infection comprising administering to a subject in need thereof, a pharmaceutically active amount of a macrocyclic CD4 mimetic according to the invention. According to certain embodiments the viral infection is an HIV infection. According to some embodiments the administration is orally. According to other embodiments the administration route is selected from the group consisting of: orally, topically, intranasally, subcutaneously, intramuscularly, intravenously, intra-arterially, intraarticulary, intralesionally or parenterally.

The present invention provides, according to yet another aspect, use of a pharmaceutical composition comprising a macrocyclic CD4 mimetic for prevention, alleviation or treatment of a viral infection. According to certain embodiments the viral infection is an HIV infection. According to some embodiments the CD4 mimetic is orally bio-available. According to yet other embodiments, the CD4 mimetic is used in a formulation suitable for oral administration.

Use of a macrocyclic molecule according to the invention for preparation of a medicament for prevention or treatment of viral infection is also within the scope of the present invention.

According to certain embodiments the viral infection is HIV infection. According to some embodiments the medicament is a CD4 macrocyclic mimetic formulated for oral administration.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a scheme describing the synthesis of the macrocyclic compounds MC-1 (Formula IV), SC-1 (Formula V) and CG-1 (Formula III).

FIG. 2 describes inhibition of HIV-1 infection in MAGI HeLa cells evaluated following treatment with 10 μM (grey) and 11 μM (dots) of the macrocyclic CD4 mimetics denoted MC-1, SC-1 and CG-1.

FIG. 3 shows inhibition of HIV-1 infection by the compound CG-1 in a concentration dependent manner in MAGI HeLa cells.

FIG. 4 depicts plasma concentrations of the macrocyclic compound CG-1, plotted against time after IV bolus and PO administration to conscious Wistar rats (n=5 in each group, values are average±SEM).

FIG. 5 represents permeability coefficients of the macrocyclic CD4 mimetics in Caco-2 model. (Values shown are mean Papp±SEM, n=3. ** P<0.01).

FIG. 6 represents permeability coefficients of the macrocyclic CD4 mimetics in the ex-vivo Ussing model. (Values shown are mean Papp±SEM, n=3. ** P<0.01).

FIG. 7 represents permeability coefficients of the compound CG-1 in the ex-vivo Ussing model. (Values shown are mean Papp±SEM, n=3. ** P<0.01).

FIG. 8 represents permeability coefficients of the compound CG-1 in Ussing model in the Jejunum, ileum and colon. (Values shown are mean Papp±SEM, n=3. ** P<0.01).

FIG. 9 depicts proportion (%) of the compound CG-1, unaffected by enzymatic degradation in the intestine, after incubation in Brush Border Membrane Vesicles (BBMV's) (Values shown as mean±SEM, n=4).

FIG. 10 represents enzymatic stability of CG-1 to rat cytochrome CYP3A4 with or without 3 μM of ketoconazole.

FIG. 11 demonstrates plasma concentration-time profiles (Mean±SEM) following oral administration of CG-1 with or without ritonavir (n=5).

### DETAILED DESCRIPTION OF THE INVENTION

The present invention provides backbone macrocyclic molecules mimicking the non-continuous active region in human CD4, which are able to interfere with the binding of viral gp120 to body's cells and therefore to inhibit retroviral penetration and infection. The present invention further provides pharmaceutical compositions comprising backbone macrocyclic CD4 mimetics. Compounds and compositions according to the present invention are metabolically stable, tissue permeable and/or oral bioavailable as demonstrated in several in-vitro, ex-vivo and in-vivo models showing stability in the GI lumen, oral bioavailability and prolonged intestinal and serum half-life. These compounds and compositions, alone or in combination with other anti-viral agents, may be used, according to the present invention for prevention and treatment of viral infection, in particular HIV infection.

### Cyclic Peptides and Backbone Cyclization

Cyclization of peptides has been shown to be a useful approach in developing diagnostically and therapeutically useful peptidic and peptidomimetic agents. Cyclization of peptides reduces the conformational freedom of these flexible, linear molecules, and often results in higher receptor binding affinities by reducing unfavorable entropic effects. Because of the more constrained structural framework, these agents are more selective in their affinity to specific receptor cavities. By the same reasoning, structurally constrained cyclic peptides confer greater stability against the action of proteolytic enzymes (Humphrey, et al., 1997, Chem. Rev., 2243-2266).

Methods for cyclization can be classified into cyclization by the formation of the amide bond between the N-terminal and the C-terminal amino acid residues, and cyclizations involving the side chains of individual amino acids. The latter method includes the formation of disulfide bridges between two ω-thio amino acid residues (cysteine, homocysteine), the formation of lactam bridges between glutamic/aspartic acid and lysine residues, the formation of lactone or thiolactone bridges between amino acid residues containing carboxyl, hydroxyl or mercapto functional groups, the formation of thioether or ether bridges between the amino acids containing hydroxyl or mercapto functional groups and other special methods. Lambert, et al., reviewed variety of peptide cyclization methodologies (J. Chem. Soc. Perkin Trans., 2001, 1:471-484).

Backbone cyclization is a general method by which conformational constraint is imposed on peptides. In backbone cyclization, atoms in the peptide backbone (N and/or C) are interconnected covalently to form a ring. Backbone cyclized analogs are peptide analogs cyclized via bridging groups attached to the alpha nitrogens or alpha carbonyl of amino acids. In general, the procedures utilized to construct such peptide analogs from their building units rely on the known principles of peptide synthesis; most conveniently, the procedures can be performed according to the known principles of solid phase peptide synthesis. During solid phase synthesis of a backbone cyclized peptide the protected building unit is coupled to the N-terminus of the peptide chain or to the peptide resin in a similar procedure to the coupling of other amino acids. After completion of the peptide assembly, the protective group is removed from the building unit's functional group and the cyclization is accomplished by coupling the building unit's functional group and a second functional group selected from a second building unit, a side chain of an amino acid residue of the peptide sequence, and an N-terminal amino acid residue.

As used herein the term “backbone cyclic peptide” or “backbone cyclic analog” refers to a sequence of amino acid residues wherein at least one nitrogen or carbon of the peptide backbone is joined to a moiety selected from another such nitrogen or carbon, to a side chain or to one of the termini of the peptide. According to specific embodiment of the present invention the peptide sequence is of 3 to 12 amino acids that incorporates at least one building unit, said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue. Furthermore, one or more of the peptide bonds of the sequence may be reduced or substituted by a non-peptidic linkage.

A “building unit” (BU) indicates an N^{α}-ω-functionalized or an C^{α}-ω-functionalized derivative of amino acids. Use of such building units permits different length and type of linkers and different types of moieties to be attached to the scaffold. This enables flexible design and easiness of production using conventional and modified solid-phase peptide synthesis methods known in the art.

In general, the procedures utilized to construct backbone cyclic molecules and their building units rely on the known principles of peptide synthesis and peptidomimetic synthesis; most conveniently, the procedures can be performed according to the known principles of solid phase peptide synthesis. Some of the methods used for producing N^{α}ω building units and for their incorporation into peptidic chain are disclosed in U.S. Pat. Nos. 5,811,392; 5,874,529; 5,883,293; 6,051,554; 6,117,974; 6,265,375, 6,355613, 6,407,059, 6,512,092 and international applications WO 95/33765; WO 97/09344; WO 98/04583; WO 99/31121; WO 99/65508; WO 00/02898; WO 00/65467 and WO 02/062819.

As used herein “peptide” indicates a sequence of amino acids linked by peptide bonds. Functional derivatives of the peptides of the invention covers derivatives which may be prepared from the functional groups which occur as side chains on the residues or the N- or C-terminal groups, by means known in the art, and are included in the invention. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl groups (for example those of seryl or threonyl residues) formed by reaction with acyl moieties. Salts of the peptides of the invention contemplated by the invention are organic and inorganic salts.

The compounds herein disclosed may have asymmetric centers. All chiral, diastereomeric, and racemic forms are included in the present invention. Many geometric isomers of double bonds and the like can also be present in the compounds disclosed herein, and all such stable isomers are contemplated in the present invention.

The term “amino acid” refers to compounds, which have an amino group and a carboxylic acid group, preferably in a 1,2- 1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanolanine), and synthetically derived α-amino acids, such as amino-isobutyric acid, norleucine, norvaline, homocysteine and homoserine. β-Alanine and γ-amino butyric acid are examples of 1,3 and 1,4-amino acids, respectively, and many others are well known to the art.

Some of the amino acids used in this invention are those which are available commercially or are available by routine synthetic methods. Certain residues may require special methods for incorporation into the peptide, and either sequential, divergent or convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” or “(D)” before the residue abbreviation.

Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

“Permeability” refers to the ability of an agent or substance to penetrate, pervade, or diffuse through a barrier, membrane, or a skin layer. A “cell permeability moiety”, a “permeability enhancing moiety” or a “cell-penetration moiety” refers to any molecule known in the art which is able to facilitate or enhance penetration of molecules through membranes. Non-limitative examples include: hydrophobic moieties such as lipids, fatty acids, steroids and bulky aromatic or aliphatic compounds; moieties which may have cell-membrane receptors or carriers, such as steroids, vitamins and sugars, natural and non-natural amino acids and transporter peptides. Examples for lipidic moieties which may be used according to the present invention: Lipofectamine, Transfectace, Transfectam, Cytofectin, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DCGS, DPPES, DCPE, DMAP, DMPE, DOGS, DOHME, DPEPC, Pluronic, Tween, BRIJ, plasmalogen, phosphatidylethanolamine, phosphatidylcholine, glycerol-3-ethylphosphatidylcholine, dimethyl ammonium propane, trimethyl ammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidylethanolamine, palmitoylhomocystiene, N,N′-Bis (dodecyaminocarbonylmethylene)-N,N′-bis((-N,N,N-trimethylammoniumethyl-ami nocarbonylmethylene)ethylenediamine tetraiodide; N,N″-Bis(hexadecylaminocarbonylmethylene)-N,N′,N″-tris((-N,N,N-trimethylammonium-ethylaminocarbonylmethylenediethylenetri amine hexaiodide; N,N′-Bis(dodecylaminocarbonylmethylene)-N,N″-bisq-N,N,N-trimethylammonium ethylaminocarbonylmethylene)cyclohexylene-1,4-diamine tetraiodide; 1,7,7-tetra-((-N,N,N,N-tetramethylammoniumethylamino-carbonylmethylene)-3-hexadecylaminocarbonyl-methylene-1,3,7-triazaheptane heptaiodide; N,N,N′,N′-tetra((-N,N,N-trimethylammonium-ethylaminocarbonylmethylene)-N′-(1,2-dioleoylglycero-3-phosphoethanolamino carbonylmethylene)diethylenetriamine tetraiodide; dioleoylphosphatidylethanolamine, a fatty acid, a lysolipid, phosphatidylcholine, phosphatidylethanolamine, phosphatidylserine, phosphatidylglycerol, phosphatidylinositol, a sphingolipid, a glycolipid, a glucolipid, a sulfatide, a glycosphingolipid, phosphatidic acid, palmitic acid, stearic acid, arachidonic acid, oleic acid, a lipid bearing a polymer, a lipid bearing a sulfonated saccharide, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy)-1-thio-b-D-galactopyranoside, digalactosyldiglyceride, 6-(5-cholesten-3b-yloxy)hexyl-6-amino-6-deoxy-1-thio-b-D-galactopyrano side, 6-(5-cholesten-3b-yloxy)hexyl-6-amino-6-deoxyl-1-thio-α-D-mannopyranoside, 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoyl-phosphatidylethanolamine; 1,2-dioleoyl-sn-glycerol; 1,2-dipalmitoyl-sn-3-succinyl-glycerol; 1,3-dipalmitoyl-2-succinylglycerol, 1-hexadecyl-2-palmitoylglycero-phosphoethanolamine, and palmitoylhomocysteine.

An “electron withdrawing group” draws electrons away from a reaction center. Electron withdrawing groups as defined herein include but are not limited to halogens, nitriles, carboxylic acids and carbonyls. Specific examples of electron withdrawing groups include but are not limited to F, Cl, Br, I, and NO₂.

### Biology

### Determination of Anti HIV Activity

The compounds of the present invention have anti-HIV activity. Such activity can be determined, for example, by the infection inhibition and β-galactosidase assays described in the examples below. Such activity can also be determined by measuring the concentration required to reduce the cytopathic effect of the virus, as described by Santosh et al., 2000, Bioorg. Med. & Chem. Lett., 10, 2505-08.

Such activity can also be determined using a plaque formation assay, and measuring the dose-dependent decrease in plaques, as described by Luedtke et al., 2002, Chembiochem, 3, 766-771. Dose-dependent activity can also be determined by measuring the decrease in HIV-1 p24 expression using ELISA.

In addition, high-throughput screening assays can be performed to identify, for example, potential inhibition of HIV integration into the host cell chromosome. See Vandergraaf et al., 2001, Antimicrobial Agents and Chemotherapy, 45:2510-16.

Permeability assays can be performed for example by the Ussing model (Koefoed-Johnsen, V., and H. H. Ussing, 1958, Acta Physiol. Scand. 42:298-308; Lane L. Clarke 2009, Am J Physiol Gastrointest Liver Physiol, 296, G1151-G1166), and as described below under “general procedures”.

Stability tests may be performed as described below under “general procedures”, for example brush border membrane vesicles stability test and microsomal test.

Conditions which may be prevented or treated with the compounds of the present invention include all conditions associated with HIV and other pathogenic retroviruses, including AIDS, AIDS-related complex (ARC), progressive generalized lymphadenopathy (PGL), as well as chronic CNS diseases caused by retroviruses, such as HIV mediated dementia and multiple sclerosis.

The compounds of the present invention can therefore be used as medicines against the above-mentioned conditions. The use comprises administering to HIV-infected subjects, or subjects at risk for HIV infection, an amount effective to combat the conditions associated with HIV and other pathogenic retroviruses, including HIV-1.

### Pharmacology

The compounds of the present invention can be formulated into various pharmaceutical forms for purposes of administration. Pharmaceutical composition of interest may comprise at least one additive selected from a disintegrating agent, binder, flavoring agent, preservative, colorant and a mixture thereof, as detailed for example in “Handbook of Pharmaceutical Excipients”; Ed. A. H. Kibbe, 3rd Ed., American Pharmaceutical Association, USA.

For example, a compound of the invention, or its salt form or a stereochemically isomeric form, can be combined with a pharmaceutically acceptable carrier. Such a carrier can depend on the route of administration, such as oral, rectal, percutaneous or parenteral injection.

A “carrier” as used herein refers to a non-toxic solid, semisolid or liquid filler, diluent, vehicle, excipient, solubilizing agent, encapsulating material or formulation auxiliary of any conventional type, and encompasses all of the components of the composition other than the active pharmaceutical ingredient. The carrier may contain additional agents such as wetting or emulsifying agents, or pH buffering agents. Other materials such as anti-oxidants, humectants, viscosity stabilizers, and similar agents may be added as necessary.

For example, in preparing the compositions in oral dosage form, media such as water, glycols, oils, alcohols can be used in liquid preparations such as suspensions, syrups, elixirs, and solutions. Alternatively, solid carriers such as starches, sugars, kaolin, lubricants, binders, disintegrating agents can be used, for example, in powders, pills, capsules or tablets.

The pharmaceutically acceptable excipient(s) useful in the composition of the present invention are selected from but not limited to a group of excipients generally known to persons skilled in the art e.g. diluents such as lactose (Pharmatose DCL 21), starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, and bentonite; disintegrants; binders; fillers; bulking agent; organic acid(s); colorants; stabilizers; preservatives; lubricants; glidants/antiadherants; chelating agents; vehicles; bulking agents; stabilizers; preservatives; hydrophilic polymers; solubility enhancing agents such as glycerin, various grades of polyethylene oxides, transcutol and glycofiirol; tonicity adjusting agents; pH adjusting agents; antioxidants; osmotic agents; chelating agents; viscosifying agents; wetting agents; emulsifying agents; acids; sugar alcohol; reducing sugars; non-reducing sugars and the like, used either alone or in combination thereof. The disintegrants useful in the present invention include but not limited to starch or its derivatives, partially pregelatinized maize starch (Starch 1500®), croscarmellose sodium, sodium starch glycollate, clays, celluloses, alginates, pregelatinized corn starch, crospovidone, gums and the like used either alone or in combination thereof. The lubricants useful in the present invention include but not limited to talc, magnesium stearate, calcium stearate, sodium stearate, stearic acid, hydrogenated vegetable oil, glyceryl behenate, glyceryl behapate, waxes, Stearowet, boric acid, sodium benzoate, sodium acetate, sodium chloride, DL-leucine, polyethylene glycols, sodium oleate, sodium lauryl sulfate, magnesium lauryl sulfate and the like used either alone or in combination thereof. The anti-adherents or glidants useful in the present invention are selected from but not limited to a group comprising talc, corn starch, DL-leucine, sodium lauryl sulfate, and magnesium, calcium and sodium stearates, and the like or mixtures thereof. In another embodiment of the present invention, the compositions may additionally comprise an antimicrobial preservative such as benzyl alcohol. In an embodiment of the present invention, the composition may additionally comprise a conventionally known antioxidant such as ascorbyl palmitate, butylhydroxyanisole, butylhydroxytoluene, propyl gallate and/or tocopherol. In another embodiment, the dosage form of the present invention additionally comprises at least one wetting agent(s) such as a surfactant selected from a group comprising anionic surfactants, cationic surfactants, non-ionic surfactants, zwitterionic surfactants, or mixtures thereof. The wetting agents are selected from but not limited to a group comprising oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate and the like, or mixtures thereof. In yet another embodiment, the dosage form of the present invention additionally comprises at least one complexing agent such as cyclodextrin selected from a group comprising but not limited to alpha-cyclodextrin, beta-cyclodextrin, betahydroxy-cyclodextrin, gamma-cyclodextrin, and hydroxypropyl beta-cyclodextrin, or the like. In yet another embodiment, the dosage form of the present invention additionally comprises of lipid(s) selected from but not limited to glyceryl behenate such as Compritol® ATO888, Compritol® ATO 5, and the like; hydrogenated vegetable oil such as hydrogenated castor oil e.g. Lubritab®; glyceryl palmitostearate such as Precirol® ATO 5 and the like, or mixtures thereof used either alone or in combination thereof. It will be appreciated that any given excipient may serve more than one function in the compositions according to the present invention.

For parenteral compositions, the carrier can comprise sterile water. Other ingredients may be included to aid in solubility. Injectable solutions can be prepared where the carrier includes a saline solution, glucose solution or mixture of both.

Injectable suspensions can also be prepared. In addition, solid preparations that are converted to liquid form shortly before use can be made. For percutaneous administration, the carrier can include a penetration enhancing agent or a wetting agent.

It can be advantageous to formulate the compositions of the invention in dosage unit form for ease of administration and uniformity of dosage. “Dosage unit form” refers to physically discrete units suitable as unitary dosages, each unit containing a pre-determined quantity of active ingredient calculated to produce the desired therapeutic effect in association with the chosen carrier.

Apart from other considerations, the fact that the novel active ingredients of the invention are peptides, peptide analogs or peptidomimetics, dictates that the formulation be suitable for delivery of these types of compounds. Although in general peptides are less suitable for oral administration due to susceptibility to digestion by gastric acids or intestinal enzymes. According to the present invention, novel methods of backbone cyclization are being used, in order to synthesize metabolically stable and oral bioavailable peptidomimetic analogs. The preferred route of administration of peptides of the invention is oral administration.

Other routes of administration are intra-articular, intravenous, intramuscular, subcutaneous, intradermal, or intrathecal.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., 2001, Curr. Opin. Chem. Biol. 5, 447). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art.

Toxicity and therapeutic efficacy of the peptides described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Those skilled in the treatment and prevention of HIV infection can determine the effective daily amount. Generally, an effective amount can be from 0.01 mg/kg to 50 mg/kg body weight and, more preferably, from 0.1 mg/kg to 10 mg/kg body weight. It may be appropriate to administer the required dose as two, three, four or more sub-doses at appropriate intervals during the day. Such sub-doses can be formulated as unit dosage forms, for instance, containing 1 to 1000 mg, more preferably 5 to 200 mg, of active ingredient per unit dosage form.

The precise dosage and frequency of administration depends on the particular compound of the invention being used, as well as the particular condition being treated, the severity of the condition, the age, weight, and general physical condition of the subject being treated, as well as other medication being taken by the subject, as is well known to those skilled in the art. It is also known that the effective daily amount can be lowered or increased depending on the response of the subject or the evaluation of the prescribing physician. Thus, the ranges mentioned above are only guidelines and are not intended to limit the scope of the use of the invention.

The combination of a compound of the invention with another anti-retroviral compound can be used. Such combination can be used simultaneously, sequentially or separately. Such anti-retroviral compounds include nucleoside reverse transcriptase inhibitors such as 3′-azido-3′-deoxythymidine (AZT), didanosine (dideoxy inosine; ddI), zalcitabine (dideoxycytidine; ddC), tenofovir, or lamivudine (3′-thia-2′-3′-dideoxycytidine; 3TC). Anti-retroviral compounds also include non-nucleoside reverse transcriptase inhibitors such as suramine, foscarnet-sodium, nevirapine, sustiva and tacrine; TIBO type compounds; α-APA type compounds; TAT inhibitors (e.g., RO-5-3335); protease inhibitors (e.g., indinavir, ritonavir, saquinovir); NMDA receptor inhibitors (e.g., pentamidine); α-glycosidase inhibitors (e.g., castanospermine); Rnase H inhibitors (e.g., dextran); or immunomodulating agents (e.g., levamisole, thymopentin).

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### General Procedures

### Chemistry General

All starting materials were purchased from commercial sources and were used without further purification. Nuclear magnetic resonance (NMR) spectra during synthesis were recorded on a Bruker AMX 300, Bruker 400 or Bruker 500 MHz spectrometer. Chemical shifts are reported downfield, relative to internal solvent peaks. Coupling constants J are reported in Hz. High Resolution Mass spectrometry (HRMS) spectra were recorded on nanospray ionization LTQ orbitrap. Matrix assisted laser desorption ionization (MALDI)-time of flight (TOF) (MALDI-TOF) Mass spectra were recorded on a PerSeptive Biosystems MALDI-TOF MS, using α-cyano-4-hydroxycinnamic acid as matrix. Thin layer chromatography (TLC) was performed on Merck aluminum sheets silica gel 60 F254. Column chromatography was performed on Merck silica gel 60 (230-400 mesh).

### Peptides Purification:

Peptides purity was determined by analytical HPLC, peptides below 95% purity were excluded from further examination (see supporting information). Analytical HPLC was performed on Vydac analytical columns (C18, 5μ, 4.6 mm×250 mm (218TP54)) using Merck-Hitachi system: Model LaChrom with a L-7100 pump, L-7200 autosampler, L-7400 UV/Vis detector and a D-7000 interface. Products were assayed at 215 and 220 nm. The mobile phase consisted of a gradient system, with solvent A corresponding to TDW with 0.1% TFA and solvent B corresponding to acetonitrile (ACN) with 0.1% TFA. The mobile phase started with 95% A from 0 to 5 min followed by a linear gradient from 5% B to 95% B from 5 to 55 min. The gradient remained at 95% B for an additional 5 min and then was reduced to 95% A and 5% B from 60 to 65 min. The gradient remained at 95% A for additional 5 min to achieve column equilibration. The flow rate of the mobile phase was 1 mL/min. Peptide purification was performed by reversed phase semi-preparative HPLC on a Merck-Hitachi 665A model equipped with a preparative pump (30 ml/min) and a high flow UV/Vis detector using semipreparative Vydac column (C18, 5μ, 10×250 (208TP510)) flow rate of the mobile phase was 4.5 mL/min. All semi preparative HPLC runs were carried out using a gradient system similar to the one used in for the analytical HPLC.

### Macrocycles Purification:

Analytical RP-HPLC were recorded at 220 nm at a flow of 1 ml/min on Merck-Hitachi system (LaChrom with a L-7100 pump, L-7200 autosampler, L-7400 UV/Vis detector and a D-7000 interface) on Phenomenex RP-18 column (C18, 5i, 4.6×75 mm (Luna)). Using the same solvent system previously described, the mobile phase started with 95% A from 0 to 5 min followed by a linear gradient from 5% B to 95% B from 5 to 17 min. The gradient remained at 95% B for an additional 4 min and then was reduced to 95% A from 21 to 25 min. The gradient remained at 95% A for additional 5 min to achieve column equilibration. Semi-preparative HPLC were recorded at 220 nm on Phenomenex RP-18 column (C18, 10μ 1250×10 mm, 110 Å (Gemini)). Using the same solvent system previously described, the mobile phase started with 95% A from 0 to 5 min followed by a linear gradient from 5% B to 35% B from 5 to 30 min, then to 95% B in 15 min, the gradient remained at 95% B for an additional 5 min and then was reduced to 95% A in 10 min. The gradient remained at 95% A for additional 5 min to achieve column equilibration.

### Peptide Synthesis

Peptides were synthesized on methylbenzhydrylamine (MBHA) resin using the combinatorial “tea bag” method (Brodsky et al., J. Immunol. 1990, 144, 3078) using Fmoc and Boc chemistries as described before (Kasher et al., J. Mol. Biol. 1999, 292, 421; Kwong et al., Structure 2000, 8, 1329). Final cleavage was performed using HF/anisole mixture or by trimethylsilyl trifluoromethane sulfonate/anisole/trifluoroacetic acid mixture. Coupling and cleavage steps were followed by free amine standard Kaiser and chloranil detection assays in order to determine the step's success (Kaiser et al., 1970, Anal. Biochem., 34:595-98; Christensen, T., 1979, Acta. Chem. Scand. B., 33:763-66).

### General Methods for Solid Phase Synthesis of Macromolecules C

Swelling: The resin was swelled for at least 2 h in DCM. Fmoc removal: The resin was treated with a solution of 20% piperidine in NMP (2×20 min), and then washed with NMP (5×2 min). HBTU coupling: Protected amino acids (1.5 equiv) were dissolved in NMP. N,N-diisopropylethylamine (DIPEA) (1.5 equiv) and 1-hydroxybenzotriazole (HOBt) (1.5 equiv) were added and the mixture was cooled to 0° C. (2-(1H-Benzotriazole-1-yl)-1,1,3,3-tetramethyluronium hexafluorophosphate) (HBTU) (1.5 equiv) was added and the mixture was pre-activated by mixing for 10 min, added to the resin, and shaken for 1 h. The resin was washed with NMP (3×2 min). Capping: The resin was treated with a solution of AC2O (10 equiv) and DIPEA (7.15 equiv) in DMF for 20 min and washed with NMP (3×2 min). HATU coupling: Fmoc protected amino acids (1.5 equiv) were dissolved in NMP, DIPEA (1.5 equiv) and 1-hydroxy-7-aza-benzotriazole (HOAt) (1.5 equiv) were added and the mixture was cooled to 0° C. (2-(7-Aza-1H-benzotriazole-1-yl)-1,1,3,3-tetramethyluronium hexafluorophosphate) (HATU) (1.5 equiv) was added and the mixture was preactivated by mixing for 10 min, added to the resin, and shaken overnight at rt. The resin was washed with NMP (3×2 min).

### Preparation of Regular Amine and Carboxyl Backbone Cyclization Building Units.

Building units were typically synthesized by procedures described in Muller et al., 1997, J. Org. Chem., 62:411-16.

### Preparation of N^{α}-(Boc-Amino Acids)N,O-Dimethyl Hydroxamates

To a solution of 0.055 mole N,O-dimethylhydroxylamine hydrochloride in 100 ml DMF were added 0.05 mole of 1, 0.055 mole PyBOP and 0.15 mole DIEA. Reaction mixture was left to stir at r.t. for 3 hours while maintaining pH at 9-10. Then 300 ml of EtOAc were added while stirring followed by 600 ml saturated NaHCO₃. The organic phase was washed with saturated NaHCO₃ (2×100 ml), water (2×100 ml), 1M KHSO₄ (2×100 ml) and water (2×100 ml), dried over Na₂SO₄ and evaporated to dryness. Product was left to dry in dessicator. TLC monitoring solvent system: EtOAc:PE (1:1). Products were obtained at 94-100% yields.

N^{α}-Boc-Arg(di-Z)N,O-dimethylhydroxamate (2a). HNMR (CDCl₃): 9.50, broad, 1H (NH); 7.33, m, 10H (Ar); 5.25, s, 2H (Ar—CH₂); 5.15, s, 2H (Ar—CH₂); 4.60, broad, 1H (Hα); 3.98, t, 2H (Hδδ′); 3.67, s, 3H (OCH₃); 3.12, s, 3H(NCH₃); 1.70, m, 4H (Hδβ′, Hγγ′); 1.40, s, 9H (Boc).

Naα-Boc-Lys(Z)N,O-dimethylhydroxamate (2b). HNMR (CDCl₃): 7.33, m, 5H (Ar); 5.10, s, 2H (Ar—CH₂); 4.67, s, 1H (Hα); 3.70, s, 3H(OCH₃); 3.20, s, 3H(NCH₃); 3.10, m, 2H (Hεε′); 1.82, m, 4H (Hββ′, Hδδ′); 1.67, m, 2H (Hγγ′); 1.43, s, 9H (Boc).

N^{α}-Boc-Pro-N,O-dimethylhydroxamate (2c). HNMR (CDCl₃): 4.60, broad, 1H (Hα); 3.71, s, 3H (OCH₃); 3.19, s, 3H (NCH₃); 1.88, m, 6H (Hββ′, Hγγ′, Hδδ′); 1.41, s, 9H (Boc). N^{α}-Boc-Tic-N,O-dimethylhydroxamate (2d). HNMR (CDCl₃): 7.15, m, 4H (Ar); 4.82, t, 1H (Hα); 3.78, s, 3H (OCH₃); 3.15, s, 5H (NCH₃, Hδδ′); 1.83, m, 2H(Hββ′); 1.45, s, 9H (Boc).

### Preparation of N^{α}-(Boc-amino acids) aldehydes

To a solution of 2 in 200 ml anhydrous THF in an ice bath and under Ar, was added portion-wise LiAlH₄ (2 eq.). When addition was over the ice bath was removed and reaction mixture was left to stir at r.t. for another hour. When reaction was over the ice bath was returned and 500 ml EtOAc was added portion-wise. Then 1200 ml 1M KHSO₄ was added and the reaction mixture was left to stir for another 30 min. Then the phases were separated and the organic phase was washed with 1M KHSO₄ (2×200 ml) and brine (2×200 ml), dried over Na₂SO₄ and evaporated to dryness. An oil was obtained. The product was kept at minus 8° C. under Ar. TLC monitoring solvent system: EtOAc:PE (1:1). Products were obtained at 55-93% yields.

N^{α}-Boc-Arg(di-Z) aldehyde (3a). HNMR (CDCl₃): 9.55, s, 1H (COH); 7.30, m, 10H (Ar); 5.15, s, 2H (Ar—CH₂); 1.72, m, 4H (Hββ′, Hγγ′); 1.37, s, 9H (Boc). N^{α}-Boc-Lys(Z) aldehyde (3b). HNMR (CDCl₃): 9.55, s, 1H (COH); 7.33, m, 5H (Ar); 5.10, s, 2H (Ar—CH₂); 4.67, s, 1H (Hα); 3.12, m, 2H (Hεε′); 1.82, m, 4H (Hββ′, Hδδ′); 1.67, m, 2H (Hγγ′); 1.43, s, 9H (Boc).
N^{α}-Boc-Pro aldehyde (3c). HNMR (CDCl₃): 9.45, s, 1H(COH); 3.50, m, 2H (Hδδ′); 1.79, m, 4H (Hββ′, H′γγ); 1.42, s, 9H (Boc).
N^{α}-Boc-Tic aldehyde (3d). HNMR (CDCl₃): 9.53, s, 1H(COH); 7.17, m, 4H (Ar); 4.67, s, 1H (Hα); 3.15, m, 2H (Hδδ′); 1.81, m, 2H (Hββ′); 1.43, s, 9H (Boc).

### Preparation of N^{α}-Alloc-Arg(Mts)-OH

To a solution of 0.57 mole H-Arg(Mts)-OH in 43 ml 4N NaOH and 6 ml iPrOH cooled in an ice bath, was added portion-wise a solution of 10 ml allylchloroformate in 20 ml 4N NaOH and 2 ml iPrOH under vigorous stirring. When addition was over the reaction mixture was left to stir under cooling for another 40 min, after which the ice bath was removed and the reaction mixture was left to stir at r.t. o.n. pH was maintained at 11 at all times. When reaction was over, 45 ml water were added, the phases were separated and the hydrous phase was washed with PE (3×30 ml). Then the hydrous phase was cooled in an ice bath and gradually acidified by concentrated HCl to pH=1. A white sticky mush is obtained. The product was extracted to EtOAc (4×50 ml). The organic phase was dried over MgSO₄, evaporated to dryness and left to dry in the dessicator. The caramel solid obtained was dissolved in CHCl₃ (200 ml), washed with 1N HCl (3×30 ml) and water (2×30 ml), dried over Na₂SO₄, evaporated to dryness and left to dry in the dessicator. A white precipitate was obtained. TLC monitoring solvent system: CHCl₃:MeOH (4:1). Yield: 73%. HNMR (CDCl₃): 6.89, s, 2H (Ar); 6.00, broad, 1H (NH); 5.82-5.95, split q, 1H (CH₂═CH—CH₂); 5.20, split d, 2H (CH₂═CH—CH₂); 4.55, d, 2H (CH₂═CH—CH₂); 2.62, s, 6H (Ar-oCH₃); 2.26, s, 2H (Ar-pCH₃).

### On-Resin Formation of Building Units

The building units were formed by reductive alkylation of Gly residues which were coupled to the solid phase by aldehydes. To bags containing the resin pre-loaded with Gly, was added a solution of 4 eq. of aldehyde 3 in NMP:MeOH (1:1) with 1% (v/v) AcOH. The peptides were shaken in this solution for 5 min. Then 4 eq. of NaBH₃CN were added and the peptides were left to shake in this reaction mixture for additional 3 hours. After completion the bags were washed with NMP:MeOH (1:1)+1% (v/v) AcOH (X1), DMF (X1), NMP (X2), DCM (X2), EtOH (X2) and finally DCM (X2).

### HIV-1 Infection Inhibition

### Growth and Maintenance of Hela Cells

Hela cells were obtained from ATCC, (Manassas, Va., USA) and then grown in 75 cm2 flasks with approximately 0.5×106 cells/flask at 37° C. in 5% CO2 atmosphere and at a relative humidity of 95%. The culture growth medium consisted of Dulbecco's Modified Eagle Medium (DMEM) supplemented with 10% heat-inactivated fetal bovine serum (FBS), 1% nonessential amino acids. The medium was replaced every other day.

### HIV-1 Infection Inhibition Assay

Titration of HIV-1 strand HXB2 in the absence or presence of the inhibitor was carried out by the multinuclear activation of a galactosidase indicator (MAGI) assay, similarly to the procedure described by Kimpton and Emerman (Virol. 66, 2232, 1992). Briefly, HeLa-CD4+beta-gal cells were transferred into 96-well plates at 15×10³ cells per well. On the following day, the cells were infected with 50 μl of serially diluted virus in the presence of 20 mg/ml of DEAE-dextran (Pharmacia, Sweden).

Two days post-infection, cultured cells were fixed with 1% formaldehyde and 0.2% glutaraldehyde in PBS. Following intensive wash with PBS, cells were stained with a solution of 4 mM potassium ferrocyanide, 4 mM potassium ferricyanide, 2 mM MgCl₂ and 0.4 mg/ml of X-Gal (Ornat, Israel). Blue cells were counted under a light microscope. According to a slightly different procedure, the assay consisted of CD4 expressing Hela P4 cells containing the β-galactosidase reporter gene placed downstream 25. the HIV-LTR promotor. These cells were seeded 24 hours prior to viral infection at a density of 5×10⁵ per cell in 24 well culture plates (TPP model by Beyneix). 50 μL of virus solution prepared from CEM infected cells' supernatant, were incubated for 1 hour with the assayed peptides at the required concentrations, at 4° C. 10 μg of anti CD4 monoclonal antibodies 13B8.2 or Leu3A were used as control. After incubation the virus solution was diluted to a total volume of 1 ml and was added to the Hela P4 cells. The cells were incubated at these conditions for 3 days after which their infection rate was assayed according to the β-galactosidase activity in the cells extract. The cells were washed well, harvested and disintegrated in a buffer containing 60 mM Na₂HPO₄, 40 mM NaH₂PO₄, 10 mM KCl, 1 mM MgSO4, 50 mM β-mercaptoethanol, 2.5 mM EDTA, 0.125% NP40, 0.125% triton, 20% glycerol, 0.2 mM PMSF and 100 U/ml approtinin. The cells extract was cleaned by centrifuge at 4° C. for 15 min at 13,000 rpm. β-galactosidase activity was determined by incubation of 150 μL of total cell extract at 37° C. for 2 hours in the presence of 6 mM ONPG (O-Nitrophenyl-β-D-galactopyranoside) in a buffer containing 80 mM Na₂HPO₄, 1 mM β-mercaptoethanol and 10 m MMgCl₂, followed by absorption measurement at 410 nm. The β-galactosidase activity was normalized according to the total protein quantity in the assay.

### Animals.

Male Wistar rats (275±20 gr) were purchased from Harlan laboratories (Rehobot, Israel). Rats were kept in a light-controlled room (light from 7:00 to 19:00) and were maintained on laboratory chow and water ad libitum. All surgical and experimental procedures were reviewed and approved by the Animal Experimentation Ethics Committee of the Hebrew University Hadassah Medical Center, Jerusalem.

### Permeability Assays

### PAMPA

The general procedure included preparation of stock solutions (2.5-5 μM) of each peptide in DMSO and then diluting the DMSO solution with PBS to achieve a concentration of 5% DMSO. The stock solution was used as starting donor well solutions for the PAMPA (MultiScreen-IP hydrophobic plate, cat. no. MAIPN4510/Millipore). A 1% solution of lecithin in dodecane was then added to each filter well at 5 μL per well. Immediately after adding the lipid membrane, donor solutions were added to the wells. Incubation times for all peptides were 16 h, after which the acceptor was sampled and analyzed using LC-MS. The permeability values (presented as Pe) for each peptide were obtained and compared to standards. Pe was calculated according to the following equation (Wohnsland, F., Faller, B., 2001, Journal of Medicinal Chemistry 44, 923-930):

$${P}_{e} = C \times ( - ln ( 1 - \frac{{[ drug ]}_{acceptor}}{{[ drug ]}_{equilibrium}} ) \\ C = \frac{{V}_{D} \times {V}_{A}}{( {V}_{D} + {V}_{A} ) Area \times Time}$$

Where V_{A} is the acceptor side volume, V_{D} the donor side volume, Area, the effective area of the membrane exposed for diffusion (cm²), and Time, the incubation time (sec).

Interaction with the Liposome Bilayer

Vesicles consisting of DMPC/PDA (2:3 molar ratio) were prepared by dissolving all lipid constituents in chloroform/ethanol (1/1) and drying together in vacuo to constant weight. The lipid films were suspended in deionized water by probe sonication at 70° C. for 3 minutes, yielding total lipid concentration of 1 mM. The vesicle suspension was cooled to room temperature, incubated overnight at 4° C., and polymerized by irradiation at 254 nm for 30 sec, resulting in an intense blue appearance of the vesicles solutions. UV-vis measurements were performed by addition of peptides from stock solutions (0.4 mg/ml) to 60 μl vesicle suspensions consisting of 0.5 mM total lipids in 25 mM Tris-base (pH 8), dilution to 200 μl by deionized water and spectra acquisition on an Analytical ELISA-reader (Jena, Germany), using a 96 wells microplate. All measurements were performed in duplicates.

To quantify the extent of blue-to-red color transitions within the vesicle suspensions, the colorimetric response (% CR), was defined and calculated as follows
% CR=[(PB0−PBI)/PB0]□100, where PB=Ablue/(Ablue+Ared), and A is the absorbance at 640 nm, the “blue” component of the spectrum, or at 500 nm, the “red” component (“blue” and “red” refer to the visual appearance of the material, not actual absorbance). PB0 is the blue/red ratio of the control sample before induction of a color change, and PBI is the value obtained for the vesicle solution after the colorimetric transition has occurred. More reddish appearance of the vesicle suspensions indicates higher CR values. Previous studies have shown that peptides interact selectively with phospholipid domains of the mixed phospholipid/PDA assemblies and chromatic transitions of 100% PDA constructs are minimal (Satchell, D. P., 2003. J Biol Chem 278, 13838-13846).
In-vitro cell based permeability model: Caco-2

### Growth and Maintenance of Cells

Caco-2 cells were obtained from ATCC, (Manassas, Va., USA) and then grown in 75 cm² flasks with approximately 0.5×10⁶ cells/flask at 37° C. in 5% CO₂ atmosphere and at a relative humidity of 95%. The culture growth medium consisted of Dulbecco's Modified Eagle Medium (DMEM) supplemented with 10% heat-inactivated fetal bovine serum (FBS), 1% nonessential amino acids, and 2 mM L-glutamine. The medium was replaced every other day.

### Preparation of Cells for Transport Studies

For transport studies cells in a passage range of 52-60 were seeded at density of 25×10⁵ cells/cm² on untreated culture inserts of polycarbonate membrane with 0.4 μm pores and surface area of 1.1 cm². The culture inserts containing Caco-2 monolayers were placed in 24 transwell plates, 12 mm, Costar™. The culture medium was changed every other day. Over approximately 21 days, the cells grow to confluence and cover the surface of the support. During the first week, the cells proliferate and generate a monolayer. During the additional 14 days, the cells differentiate, polarize to apical (luminal) and basolateral, develop the microvilli morphology on the apical surface and increasingly express transporter proteins.

### Experimental Protocol

Transport study was initiated by medium removal from both sides of the monolayer and replacement with apical buffer (600 μl) and basolateral buffer (1500 μl), both pre-warmed to 37° C. The cells were incubated for a 30 min period at 37° C. with shaking (80 cycles/min). After the incubation period the buffers were removed and replaced with 1500 μl basolateral buffer at the basolateral side. Pre-warmed test solutions were added (600 μl) to the apical side of the monolayer. 50 μl samples were taken from the apical side immediately at the beginning of the experiment. For the duration of the experiment, the cells were kept at 37° C. with shaking. At predetermined times (30, 60, 90, 120, 150 and 180 min), 200 μl samples were taken from the basolateral side and replaced with the same volume of fresh basolateral buffer to maintain a constant volume.

### Evaluation of Monolayer Integrity

The cells were tested for their trans-epithelial electrical resistance (TEER) as a method to evaluate the polarization of the cell monolayer and formation of tight junctions. TEER values were in the range of 200-300Ω×cm². Inserts with deviational values were not used.

Mannitol and testosterone, commonly used markers for passive paracellular and transcellular permeability accordingly, were used in order to further evaluate the proper carrying out of each study.

### Data Analysis

The permeability coefficient (Papp) for each compound was calculated from the linear plot of drug accumulated vs. time, using the following equation:

Papp=dQ/dt/(C₀·A)

Where dQ/dt is the steady state rate of appearance of the drug on the receiver side, C₀ is the initial concentration of the drug on the donor side, and A is the surface area, 1.1 and 0.5 cm² for Caco-2 and Ussing chamber experiments, respectively.

### Permeability Ex-Vivo Side by Side Diffusion Chamber

Permeability experiments were performed in a modified Ussing chamber system (Physiological Instruments Inc. San-Diego, Calif.). Male Wistar rats, 250-300 g, were used. Following a midline incision, 25 cm of small intestine was removed and placed in ice-cold Ringer bicarbonate buffer (NaCl 6.54 gr, KCl 0.37 gr, CaCl₂×2H₂O 0.18 gr, MgCl₂×6H₂O 0.24 gr, NaHCO₃ 2.1 gr, Na₂HPO₄ 0.23 gr, NaH₂PO₄ 0.05 gr in 1000 ml). All buffer solutions were freshly prepared and equilibrated to pH 7.4. The jejunal portion of the small intestine (10-15 cm distal to the pylorus) was used. Sections containing Peyer's patches were not used in these studies. The individual segments were obtained and underlying muscularis was removed from the serosal side of the tissue before mounting. The exposed tissue surface area was 0.5 cm² and fluid volume in each half-cell was 3 ml. The system was preheated to 37° C. Modified Ringer buffers were added to the serosal and the mucosal sides (mucosal modified Ringer buffer contained 10 mM mannitol, and serosal modified Ringer buffer contained 8 mM D-glucose and 2 mM mannitol). The tissue oxygenation and the solution mixing were performed by bubbling with 95% O₂-5% CO₂. The system was equilibrated for 30 min. The permeability experiments continued for 150 min, samples were withdrawn at predetermined times. The sampled volume was replaced by blank (non-compound containing) buffer to maintain sink conditions. The integrity of epithelial tissue was monitored by measuring TEER values throughout the experiment. Any tissue with values <30 Ωcm² was discarded before the start of the experiment. Generally, TEER values were 60-110 Ωcm² and remained steady throughout the experiment.

### Metabolic Stability Models:

### Brush Border Membrane Vesicles Stability

Brush border membrane vesicles (BBMVs) were prepared from combined duodenum, jejunum, and upper ileum by a Ca⁺² precipitation method (Peerce, B. E., 1997., Biochim Biophys Acta 1323, 45-56, Peerce et al. 2003., Biochem Biophys Res Commun 301, 8-12). The intestines of 5 male Wistar rats, 200-250 gr, were rinsed with ice cold 0.9% NaCl and freed of mucus; the mucosa was scraped off the luminal surface with glass slides and put immediately into buffer containing 50 nM KCl and 10 mM Tris-HCl (pH 7.5, 4° C.) and then homogenated (Polytron PT 1200, Kinematica AG, Switzerland). CaCl₂ was added to a final concentration of 10 mM. The homogenate was left shaking for 30 min at 4° C. and then centrifuged at 10,000 g for 10 min. The supernatant was then centrifuged at 48,000 g for 30 min and an additional two purification steps were undertaken by suspending the pellet in 300 mM mannitol and 10 mM Hepes/Tris (pH 7.5) and centrifuging at 24,000 g/hr. Purification of brush border membranes was assayed using the brush border membrane enzyme markers gamma-glutamyl transpeptidase (GGT), leucine amino peptidase (LAP) and alkaline phosphatase. During the course of these studies, enrichment in brush border membrane enzymes varied between 13- and 18-fold.

### Metabolic Stability Protocol

The enzymatic reaction was performed as previously reported (Ovadia et al., 2009, Bioorg Med Chem 18, 580-589). Briefly, 2 μM stock solutions of the compounds were diluted with serum or purified BBMVs to final 0.5 μM. During incubation at 37° C. samples were taken at fixed time points. The enzymatic reaction was stopped by adding 2:1 v/v of ice cold acetonitrile or methanol and centrifuged (4000 g, 10 min) before analysis.

### Microsomal Stability

The oxidative metabolism was evaluated in pooled liver microsomes of rats and humans with or without 3 μM of ketoconazole (CYP3A4 inhibitor) (Yang et al., 2005, Biopharm Drug Dispos 26, 387-402). The microsomes were purchased from BD Biosciences (Woburn, Mass.). The concentrations of cytochrome P450 (CYP) enzymes in these preparations were 0.48, 0.79 nmol/mg protein in humans and rats respectively. The incubation mixture (3 ml) was prepared in triplicate for each species in a 0.1 m potassium phosphate buffer (pH 7.4) containing 1 μM of substrate, 0.5 μM CYP enzymes, 1.8 mM glucose-6-phosphate and 0.4 units/ml glucose-6-phosphate dehydrogenase. After a 5 min preincubation at 37° C., the reaction was initiated by adding β-NADPH (0.15 mM final concentration). Aliquots of 0.25 ml were taken at 0, 15 and 30 min and placed into 3 volumes of icecold acetonitrile (containing an internal standard) to terminate the reaction. The samples were vortexed and centrifuged at 10000 RPM for 8 min to collect supernatant for sample analysis.

### EXAMPLES

### Example 1

### Solid-Phase Synthesis SC-1 (Formula IV, method A)

Fmoc-Rink-amide MBHA resin was washed with NMP and left 2 h for swelling. Fmoc group was removed using 20% piperidine solution and then resin was washed with NMP (3×5 min). Fmoc-Gly-OH was coupled using HBTU activation followed by washing with NMP (3×5 min). The Fmoc group was removed, and the resin was washed with NMP (3×5 min). Fmoc-L-Arg(Alloc)2—CHO (4 equiv), dissolved in NMP/MeOH solution containing 1% AcOH, was added to the resin followed by the addition of NaBH3CN (4 equiv) and left to stir for 4 h. Resin was washed with NMP/MeOH (1×5 min), MeOH (1×5 min), 1% AcOH/water (1×5 min), 10% water in MeOH (1×5 min), MeOH (1×5 min), NMP (1×5 min), DCM (1×5 min) and NMP (3×5 min). Boc-L Phenylalanine-OH was coupled using HATU activation overnight followed by washing with NMP (3×5 min). Boc was removed using the recently published procedure 21. Resin was treated with 0.05M SiC14 in DCM (dry) (2×15 min) followed by washing with DCM (1×5 min), DMF (1×5 min), 20% MeOH/DMF (1×5 min), 1% DIPEA/DMF (2×5 min) and NMP (3×5 min). Pimelic acid (10 equiv) was pre-activated with DIC (10 equiv) in NMP and was added to the resin followed by addition of 4-(Dimethylamino)pyridine (DMAP) (1 equiv) left for 3 h then washed with NMP (3×5 min). The Fmoc group was removed, resin was washed with NMP (3×5 min) then treated with a solution of PyClock (6 equiv) and DIPEA (14 equiv) in NMP for 4 h (×2). Resin was washed with NMP (2×5 min), DCM (2×5 min) and MeOH (2×5 min) then dried under vacuum. Resin was treated with Pd(PPh3)4(0) (1:1 weight equiv) in NMM/AcOH/DCM(dry)(2.5/2.5/95%) solution for 2 h in dark then washed with 0.5% DIPEA in NMP (3×5 min), 0.5% sodium diethyldithiocarbamate trihydrate in NMP (5×2 min), NMP (2×2 min), DCM (2×2 min), MeOH (2×2 min) and dried in vacuum. Crude was cleaved from the resin by treatment with TFA/triisopropylsilane (TIPS)/TDW (92.5/5/2.5%) solution for 2.5 h. The solution was separated by filtration and the resin was rinsed with neat TFA. The TFA was evaporated to give crude oil that was dissolved in ACN:TDW 1:1 solution and lyophilized. Crude was purified on semi preparative HPLC as described above, collected peaks were analyzed using analytical HPLC and pure compounds (over 95% purity were used for biological screening).

### Results for SC-1:

Prepared from 200 mg of Fmoc-Rink MBHA resin. Yield: 1.2 mg. HPLC purity >95%. Rt 9.73. HRMS (Orbitrap-ESI): exact mass calcd for C₂₄H₃₈N₇O₄ 488.2980 (MH+). Found 488.2968.

### Example 2

### Solid-Phase Synthesis MC-1 (Formula V, method B)

Fmoc-Rink-amide MBHA resin was washed with NMP and left 2 h for swelling.

Fmoc group was removed and resin was washed with NMP (3×5 min). Fmoc-Gly-OH was coupled using HBTU activation followed by washing with NMP (3×5 min). The Fmoc group was removed and resin was washed with NMP (3×5 min). Alloc-L-Tyr(tBu)-CHO (4 equiv) in 1% AcOH in NMP/MeOH was added to the resin followed by the addition of NaBH3CN (4 equiv) and left to stir for 4 h. Resin was washed with NMP/MeOH (1×5 min), MeOH (1×5 min), 1% AcOH/water (1×5 min), 10% water in MeOH (1×5 min), MeOH (1×5 min), NMP (1×5 min), DCM (1×5 min) and NMP (3×5 min). Fmoc-L-Arg(2,2,4,6,7-pentamethyldihydrobenzofuran-5-sulfonyl (pbf))-OH was coupled using HATU activation overnight followed by washing with NMP (3×5 min). Fmoc group was removed and resin was washed with NMP (3×5 min). Pimelic acid (10 equiv) was pre-activated with DIC (10 equiv) in NMP and was added to the resin followed by addition of DMAP (1 equiv) left for 3 h. Resin was washed with NMP (2×5 min), DCM (2×5 min) and MeOH (2×5 min) then dried under vacuum. Alloc was removed by treatment with Pd(PPh3)4(0) (0.5 equiv) in NMM/AcOH/DCM(dry)(2.5/2.5/95%) solution for 2 h in dark, then washed with 0.5% DIPEA in NMP (3×5 min), 0.5% sodium diethyldithiocarbamate trihydrate in NMP (5×2 min), NMP (2×2 min), DCM (2×2 min), MeOH (2×2 min) and dried in vacuum. The Fmoc group was removed, resin was washed with NMP (3×5 min) then treated with a solution of PyClock (6 equiv) and DIPEA (14 equiv) in NMP for 4 h (×2). Resin was washed with NMP (2×5 min), DCM (2×5 min) and MeOH (2×5 min) then dried under vacuum. Crude was cleaved from the resin by treatment with TFA/TIPS/TDW (92.5/5/2.5%) solution for 2.5 h. The solution was separated by filtration and the resin was rinsed with neat TFA. The TFA was evaporated to give crude oil that was dissolved in ACN:TDW 1:1 solution and lyophilized. Crude was purified on semi preparative HPLC as described above, collected peaks were analyzed using analytical HPLC and pure compounds (over 95% purity were used for biological screening).

### Results for MC-1:

Prepared from 200 mg of Fmoc-Rink MBHA resin. Yield: 1.2 mg. HPLC purity >95%. Rt 8.79. HRMS (Orbitrap-ESI): exact mass calcd for C₂₄H₃₈N₇O₅ 504.2929 (MH+). Found 504.2919.

### Example 3

### Solid-Phase Synthesis of CG-1 (Formula III, method C)

Fmoc-Rink-amide MBHA resin was washed with NMP and left 2 h for swelling. The Fmoc group was removed and resin was washed with NMP (3×5 min). Fmoc-Gly-OH was coupled using HBTU activation followed by washing with NMP (3×5 min). The Fmoc group was removed and resin was washed with NMP (3×5 min). Alloc-L-Phe-CHO (4 equiv) in 1% AcOH in NMP/MeOH was added to the resin followed by the addition of NaBH3CN (4 equiv) and left to stir for 4 h. Resin was washed with NMP/MeOH (1×5 min), MeOH (1×5 min), 1% AcOH/water (1×5 min), 10% water in MeOH (1×5 min), MeOH (1×5 min), NMP (1×5 min), DCM (1×5 min) and NMP (3×5 min). Fmoc-L-Arg(pbf)-OH was coupled using HATU activation overnight followed by washing with NMP (3×5 min), DCM (2×5 min) and MeOH (2×5 min) then dried under vacuum. Alloc was removed by treatment with Pd(PPh3)4(0) (0.5 equiv) in NMM/AcOH/DCM(dry)(2.5/2.5/95%) solution for 2 h in dark, then washed with 0.5% DIPEA in NMP (3×5 min), 0.5% sodium diethyldithiocarbamate trihydrate in NMP (5×2 min), NMP (2×2 min), DCM (2×2 min), MeOH (2×2 min) and NMP (2×3 min). Pimelic acid (10 equiv) was pre-activated with DIC (10 equiv) in NMP and was added to the resin followed by addition of DMAP (1 equiv) left for 3 h, then washed with NMP (3×5 min) and the Fmoc group was removed using a solution of 10% 1,8-diazabicyclo[5.4.0]undec-7-ene (DBU) in NMP (2×½ h). The resin was washed with NMP (3×5 min) then treated with a solution of PyClock (6 equiv) and DIPEA (14 equiv) in NMP for (2×4 h). The resin was washed with NMP (2×5 min), DCM (2×5 min) and MeOH (2×5 min) then dried under vacuum. Crude was cleaved from the resin by treatment with TFA/TIPS/TDW (92.5/5/2.5%) solution for 2.5 h. The resin was filtered and rinsed with neat TFA. The TFA solution was evaporated to give a crude oil that was dissolved in acetonitrile:TDW 1:1 solution and lyophilized. The crude product was purified on semi preparative HPLC as described above, collected peaks were analyzed using analytical HPLC and pure compounds (over 95% purity) were used for biological screening.

### Results for CG-1:

Prepared from 200 mg of Fmoc-Rink MBHA resin. Yield: 1.2 mg (2.1%). Analytical HPLC purity >95%. Rt 9.71. HRMS (Orbitrap-ESI): exact mass calcd for C₂₄H₃₈N₇O₄ 488.2980 (MH+). Found 488.2976.

### Example 4

### Design and Synthesis of Several Backbone Cyclic CD4 Mimetics

A rational design method for conversion of a non-continuous binding site in human CD4, in combination with backbone cyclization methods, was used to identify macrocyclic molecules capable of interfering with the binding of viral gp120 with human CD4. A library of backbone cyclic peptides library preserve the two crucial pharmacophores of the CD4 active site (Phe43 and Arg59) was previously synthesized (WO 2006/137075). To obtain an active backbone cyclic CD4 mimetic, four amino acids comprising the turn around the Phe43 were preserved and the Arg moiety was introduced as part of the bridge. Optimization of the distance between the Arg and Phe residues was achieved by gradual addition of one or two methylenes to both sides of the bridge:

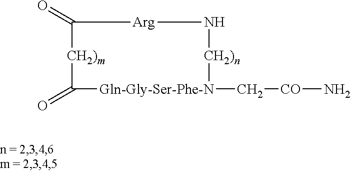

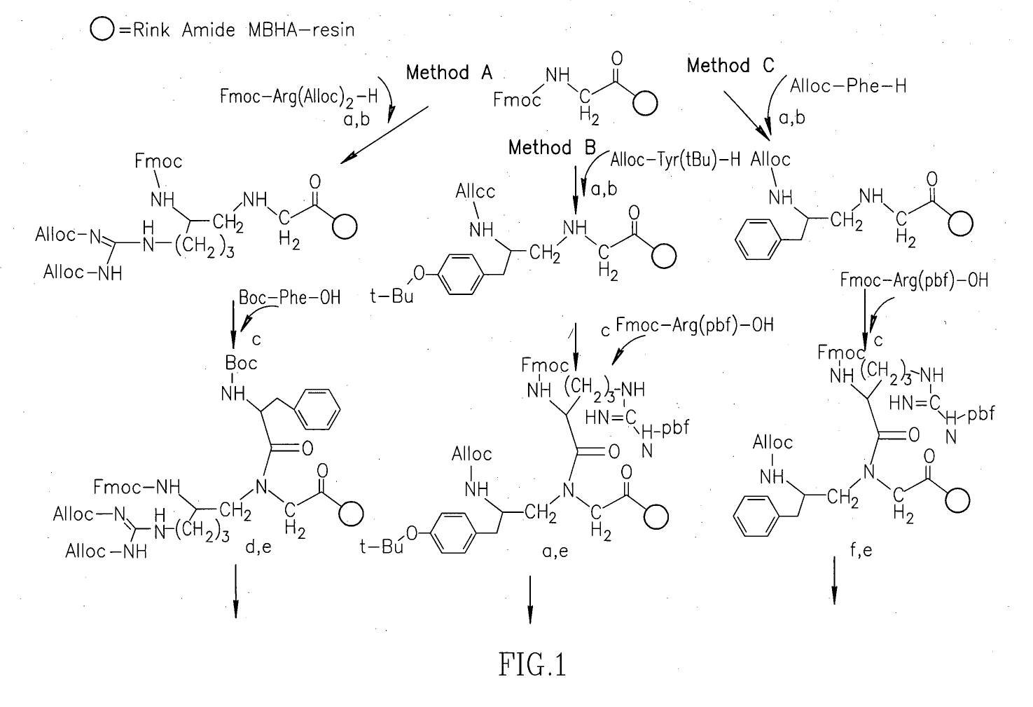

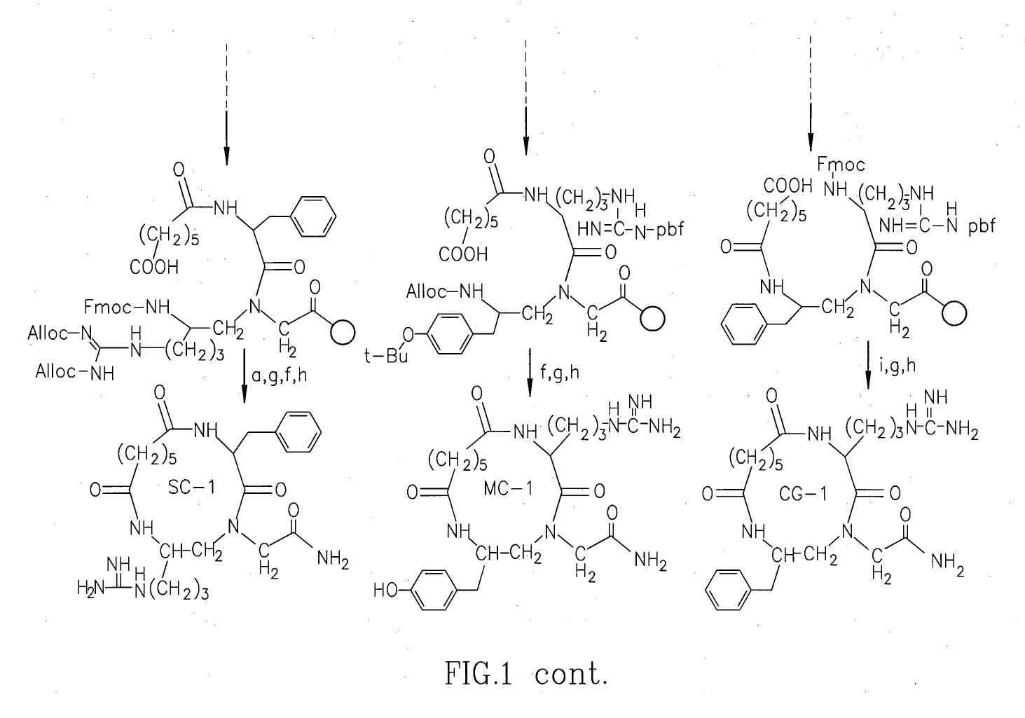

These systematic changes enabled to partially scan the conformational space of the scaffold, thus leading to the desired bioactive conformation. The next step was to reduce the distance between the important pharmacophores namely, Phe and Arg on the m side of the most potent backbone cyclic inhibitor C-2-2 (n=2, m=3). The effect of backbone cyclic peptides on HIV-1 propagation was studied by using MAGI cells, which express the β-galactosidase gene under transcription activator region regulation (Kimpton, J.; Emerman, M., 1992, J Virol. 66, 2232). Mock or HIV-1 infected cultured cells show that 100 μM of the cyclic peptide C-2-2 reduced the viral infectivity by over 80%. In order to reduce the size of the potential HIV-1 inhibitors, three amino acids from the Phe region (Gln40, Gly41 and Ser42) were replaced with a diamide linker (pimelic acid). Furthermore, since the proximity of the Arg and Phe pharmacophores is critical for the anti HIV-1 activity, it was decided to shorten the distance between these two crucial pharmacophores. The distance between the Arg guanidine and the Phe aromatic moiety in C-2-2 is 12 atoms. By inserting the guanidine or the phenylic moiety as part of the bridge methylene linker the distance between the two functional groups could be shorten to only 9 atoms. Three molecules, CG-1 (Formula III), SC-1 (Formula V) and MC-1 (Formula IV), with molecular weights of about 500 g/mol were synthesized on solid support. The three molecules share a similar scaffold comprising fourteen atoms in the ring and the same chirality of the side chains as described in FIG. 1.

All macrocyclic scaffolds were constructed using similar synthetic steps. The most challenging step was the on-resin reductive amination. Three aldehydes were synthesized in solution (see supporting information). Aldehydes Fmoc-L-Arg(Alloc)₂-H and Alloc-L-Tyr(t-Bu)-H were produced by oxidation of the corresponding alcohols using Dess-Martin periodinane as described previously (Bondebjerg et al., 2002, J Am Chem Soc 124, 11046, Myers et al., 2000, Tetrahedron Lett. 41, 1359).

Alloc-L-Phe-H was prepared by reduction of the corresponding Weinreb amide as described (Ede et al., J Pept Sci., 2000, 6, 11, Nahm, S. & Weinreb, S. M., 1981, Tetrahedron Lett., 22, 3815). The alpha nitrogen of Rink-Amide 4-Methylbenzhydrylamine (MBHA) resin bound glycine was treated with the above aldehydes and reduced with NaCNBH3 to give the corresponding secondary amine. A protected amino acid was coupled to the obtained secondary amine using either bis(trichloromethyl)carbonate (BTC) or 2-(7-Aza-1H-benzotriazole-1-yl)-1,1,3,3-tetramethyluronium hexafluorophosphate (HATU) as coupling additive. The use of milder reagents proved insufficient for complete coupling. After coupling, the temporary protecting group (Fmoc on MC-1, Boc on SC-1 and allyloxycarbonyl (Alloc) on CG-1) were removed. Selective Boc deprotection in the synthesis of SC-1 was a challenge. Boc is commonly used as permanent protection in Fmoc based solid phase synthesis and its removal was considered as non-orthogonal to the appropriate resin. Sivanandaiah et al. (Int J Pept Protein Res., 1995, 45, 377) reported iodotrichlorosilane as an efficient agent for Boc deprotection₂₀. However, this method proved too cumbersome to be used routinely and was abandoned after few attempts. A recently published procedure described by Freeman and Gilon (Synlett 2009, 2097) was therefore used for the selective removal of Boc from peptide-bound to Rink Amide MBHA resin. This procedure proved to be mild enough to allow selective Boc removal without major reduction in overall yield. Pimelic acid was attached to the primary amine using N,N′-Diisopropylcarbodiimide (DIC) for activation. The second temporary protecting group was removed according to standard procedures (Alloc for MC-1, Fmoc for CG-1 and SC-1). However, incomplete Fmoc removal from the Arg moiety in CG-1 synthesis using standard conditions occurred. Therefore, the more reactive base, 1,8-diazabicyclo[5.4.0]undec-7-ene (DBU), was used to ensure complete removal of Fmoc before the cyclization step. Cyclization was performed using the new coupling reagent 6-Chloro-Benzotriazole-1-yl-oxy-tris-Pyrrolidino-Phosphonium Hexafluorophosphate (PyClock). For MC-1 and CG-1 a simple cleavage procedure was used for consecutive removal of the remaining semi-permanent protecting groups and the macrocycles from the resin. For SC-1, Alloc removal from the guanidine moiety was performed prior to cleavage. The macrocyclic scaffolds synthesized, MC-1, SC-1 and CG-1 possess an active pharmacophore as part of the bridge.

Introduction of functional linkers by on-resin reductive amination procedure (extension of the works previously published (Bondebjerg et al., 2002, J Am Chem. Soc. 124, 11046, Hurevich et al., 2007, Heterocycles 73, 617, Qvit et al., 2008, J Comb Chem 10, 256), was utilized to synthesize the macrocycles on solid support. Adopting these methods minimize the amount of synthetic steps involved in precursors' preparation.

Previous studies demonstrated that the role of CD4 Phe43 is crucial for gp120 binding and its replacement with other amino acids including tyrosine resulted in dramatic decrease in binding ability (Moebius et al. 1992, J Exp Med, 176, 507). To test whether CG-1 binds gp120 in the same manner as CD4, the phenyl moiety in CG-1 was replaced with a phenol. The resulting molecule, MC-1, has exactly the same scaffold, structure and pharmacophores topology as CG-1 but possesses a hydroxyl on the para position of the phenyl moiety replacing hydrogen. CG-1 and SC-1 are structural regioisomers having the same scaffold that preserve the original CD4 active pharmacophores but differ in the position of the functional groups (FIG. 1).

### Example 5

### Biological Activity of Small Molecule Macrocyclic HIV-1 Infection Inhibitors

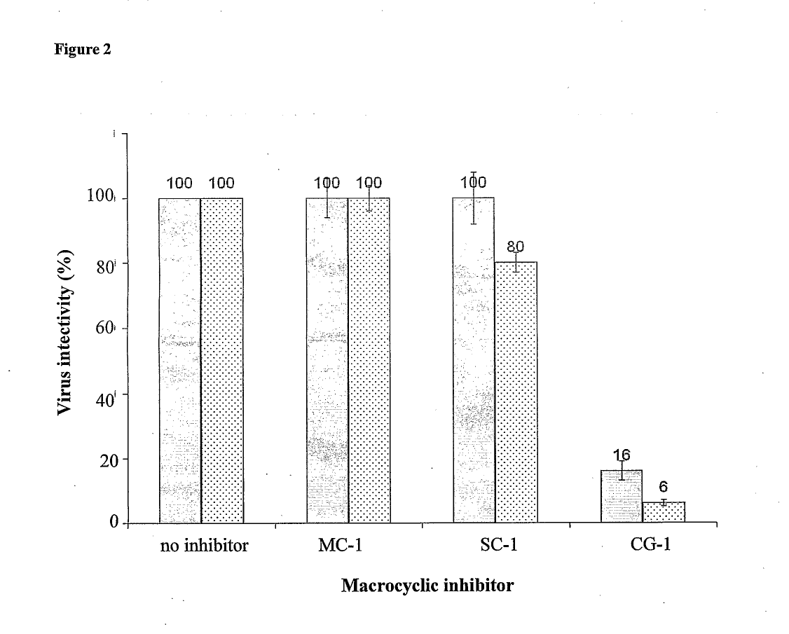

The effect of MC-1, SC-1 and CG-1 on HIV-1 propagation was studied in MAGI assay. It was shown that MC-1 did not reduce the infectivity of HIV-1 in cultured cells at the μM range (FIG. 2) indicating that the CD4:gp120 interaction was not interrupted by MC-1. The addition of the hydroxyl group to Phe43 on CD4 disrupts low energy interactions with the gp120 hydrophobic pocket.

CG-1 and SC-1 are structural regioisomers having the same scaffold that preserve the original CD4 active pharmacophores but differ in the position of the functional groups (FIG. 1). While SC-1 shows only weak inhibitory effect on HIV-1 infection (inhibits virus infection in the high μM range), CG-1 inhibits more then 80% of viral infection in the low μM range. The difference between the CG-1 and SC-1 isomers in HIV-1 infection inhibition indicates that the potency is dictated not only by the nature of the pharmacophores but also influenced by the correct orientation of the active moieties.

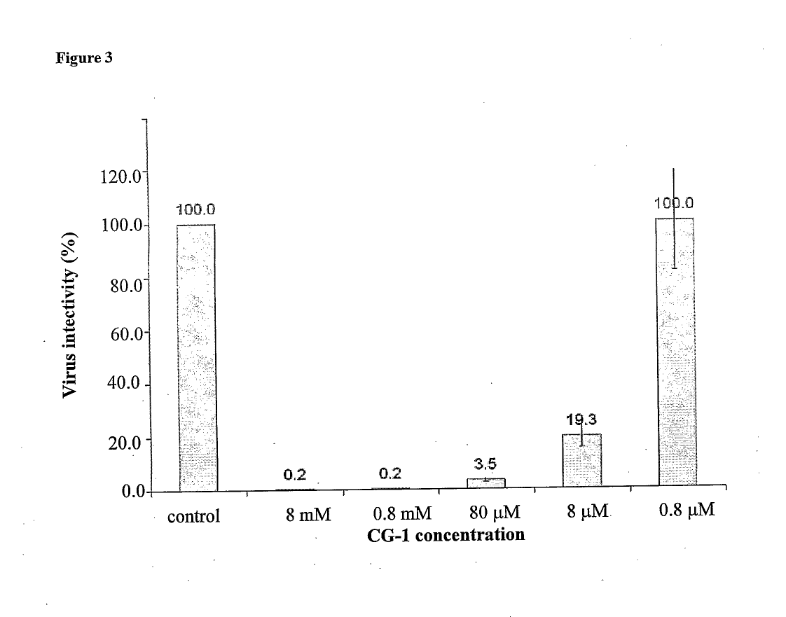

The effect of CG-1 on virus infection was further studied by three independent assays that reaffirmed in a concentration dependent manner that CG-1 has low μM activity. FIG. 3 as a preventative of such assays, indicates the number of infectious units of CD4 enriched HELA cells in presence of several CG-1 concentrations.

### Example 6

### Biological Activity of CG-1 on Pseudo Typed Virus Infection

CG-1 inhibited HIV-1 infection by blocking the viral gp-120 and therefore preventing the gp-120 attachment to CD-4. In order to check specificity pseudo typed viruses that express HIV-1 genes without the envelope proteins and express VSV-gp (vesicular stomatitis virus-glycoprotein) instead of gp-120 on the viral envelope were prepared. Infection inhibition of these viruses was checked in MAGI assay in presence of CG-1. CG-1 did not have any effect on infection inhibition of these viruses.

### Example 7

### Pharmacokinetic Profile of CG-1 Following IV and PO Administration

In 2004, Dahno and Co-workers (Third International and Twenty-Eight European Peptide Symposium; Martin et al., Eds.; Prague, Czech Republic, 2004) evaluated the reasonable size for drug candidates that aim to block transmembrane proteins interaction. The work was based on several commercially available drugs and suggests that the optimal distance between the important residues is 10-15 Å. The size of the CD4 mimetic CG-1 falls well within this criteria and has drug-like structure and characteristics. The potential of this and similar molecules as drug candidate was examined by studying their oral bioavailability properties.

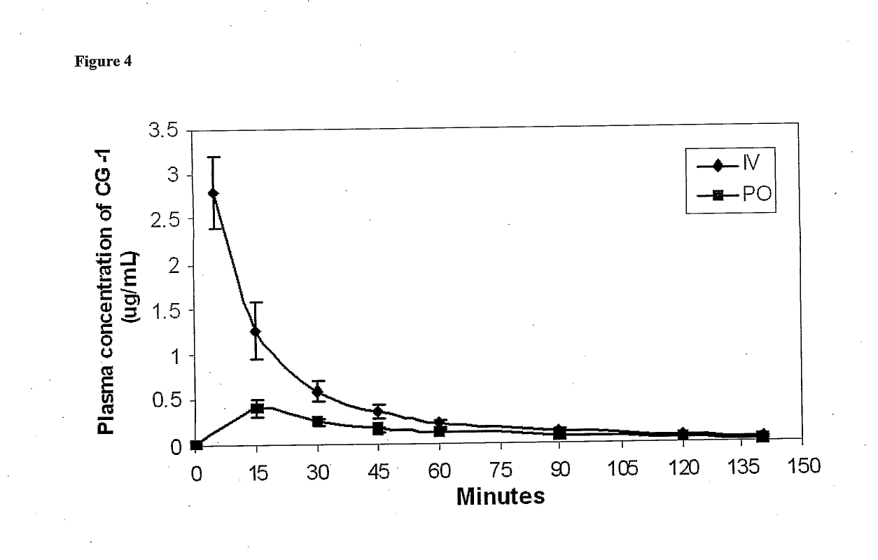

The pharmacokinetic profile of CG-1 was studied following intravenous (IV) and per-os (PO) administration to rats. Studies were performed in conscious Wistar male rats. An indwelling cannula was implanted into the left jugular vein 24 hours before the pharmacokinetic experiment to allow full recovery of the animals from the surgical procedure. Animals (n=5) received an intravenous (IV) bolus dose of 1 mg/kg of CG-1 or 10 mg/kg by oral gavage (n=5), CG-1 was dissolved in water. Blood samples (with heparin, 15 U/ml) were collected at several time points up to 24 h after. CG-1 administration. Plasma was separated by centrifugation (4000 g, 5 min, 4° C.) and stored at −70° C. pending analysis. A non-compartmental pharmacokinetic approach was used to compare pharmacokinetic profiles obtained following different modes of administration and to calculate bioavailability values. All calculations were performed utilizing WinNonlin® 5.0.1 software (Pharsight Corporation, Mountain View, Calif., USA). FIG. 4 shows plasma concentrations of CG-1 plotted against time scale after IV bolus and PO administration to conscious Wistar rats (n=5 in each group, values are average±SEM). The pharmacokinetic parameters derived from administration of CG-1 to rats are depicted in Table 1.

TABLE 1
Pharmacokinetic properties of CG-1 after administration to Wistar rats

| | Cmax | Tmax | AUC (min × | | Vss | T½ | Oral |
|---|---|---|---|---|---|---|---|
| | (ng/mL) | (min) | ng/mL) | CI (mL/min/kg) | (L/kg) | (min) | bioavailability |
| PO | 866 ± 76 | 18 | 21770 ± 63 | 14.53 ± 0.33 | 0.6 | 73 | ~10% |
| IV | 102 ± 19 | 5 | | | | | |

Non-compartmental pharmacokinetic analysis was performed using WinNonlin software, standard.

As shown in Table 1, CG-1 half-life was about 73 minutes. This is a relatively long half life in comparison to native peptides. The calculated oral bioavailability (10%) further strengthened the findings of good ex-vivo permeability. The relatively high volume of distribution (Vss: 0.6 L/kg) provides an indirect indication of the restricted ability to cross biological membranes.

Backbone cyclization and macrocyclization was used according to the present invention to design small macrocyclic inhibitors that mimic the non-continuous active region in HIV-1 CD4, and inhibit HIV-1 CD4:gp120 interaction. The unique protecting group manipulation and, in particular, the novel orthogonal Boc deprotection procedure used here was essential for the synthesis of the scaffolds and can be adopted as strategy for the preparation of other molecules on solid support. The macrocyclic compound denoted CG-1 exhibits high stability and superior oral bioavailability, and inhibits viral infection in cells in the low micromolar range making it is an attractive lead for use as inhibition of HIV-1 infection and for further drug development.

### Example 8

### Intestinal Permeability In-Vitro Tested in the Caco-2 Model

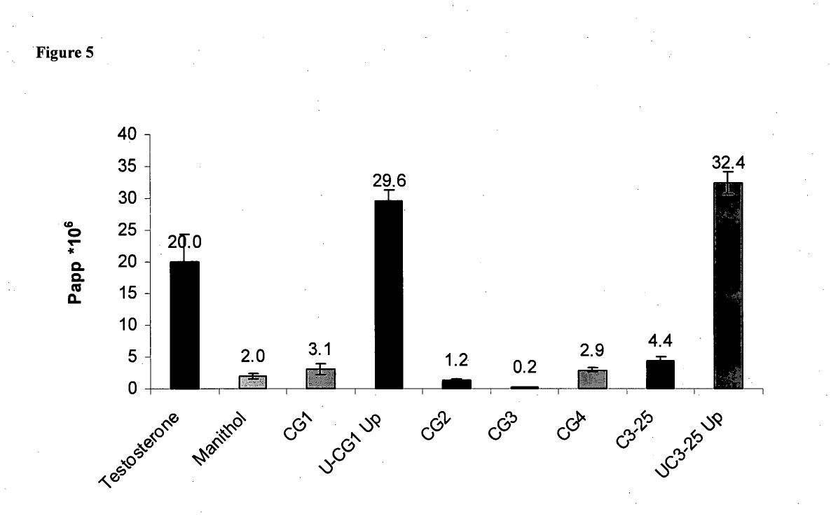

In the Caco-2 model the permeability coefficient values (Papp) of the macrocyclic analogs was relatively low, except the urea analogs UCG-1-Up and UC3-25-Up (Formulae IX and X respectively), which have better Papp values than testosterone (the passive diffusion marker for transcellular transport) as shown in FIG. 5, and by far better than the Papp value of mannitol, the marker for paracellular transport.

### Example 9

### Permeability in the Ex-Vivo Model

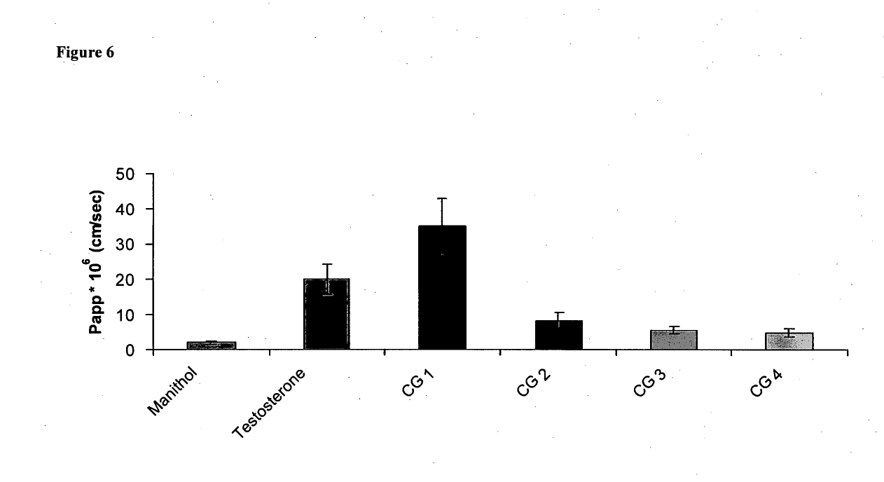

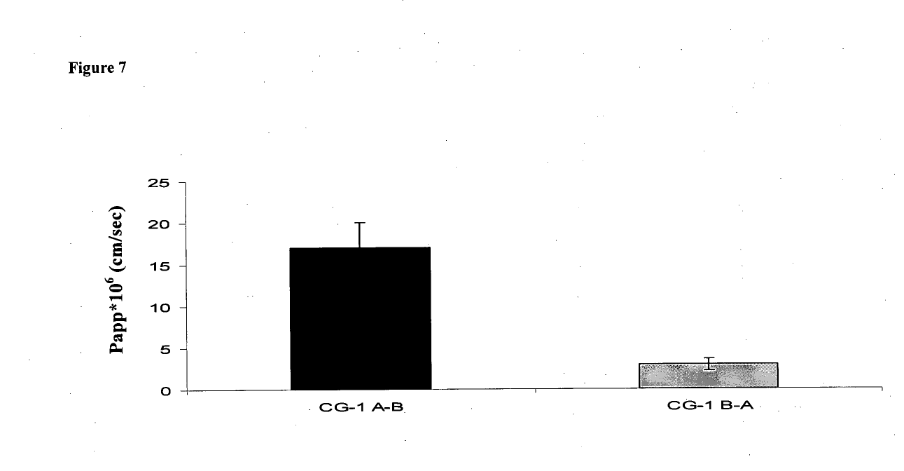

In the Ex-vivo model the permeability coefficient (Papp) of CG-1 was significantly higher than the other analogs as shown in FIG. 6. This indicates that the permeability of CG-1 is transport mediated. More ex-vivo studies were performed in order to study the transport mechanism through membrane of CG-1. As shown in FIG. 7. Papp A-B of CG-1 was significantly higher than Papp B-A (1.47*10-5, 2.86*10-6).

### Example 10

### Absorption Window of CG-1 in Ex-Vivo Model

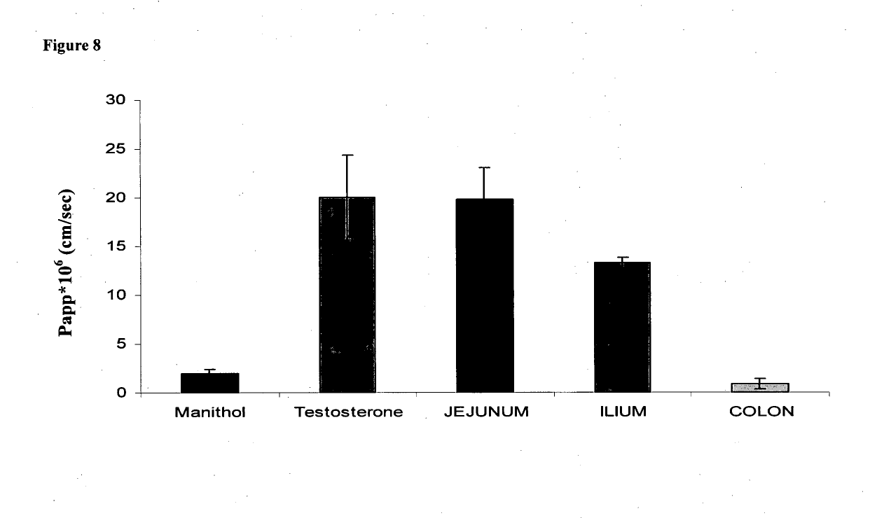

The absorption window of CG-1 was tested in the ex-vivo model. The transport of CG-1 was significantly higher in the jejunum, a region rich with transporters. The transport was significantly lower in the distal regions where the transport mediated systems are poor. The results are shown in FIG. 8 and indicate that CG-1 permeate via transporters.

### Example 11

### Metabolic Stability of CG-1

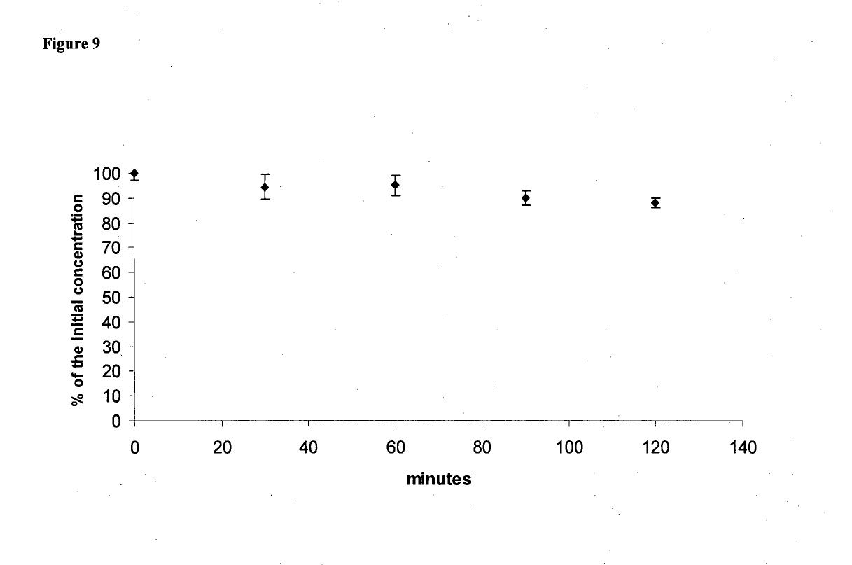

The stability of CG-1 to enzymatic degradation was tested in the intestine, after incubation in Brush Border Membrane Vesicles (BBMV's) as detailed above. The results showed in FIG. 9 indicate that CG-1 is metabolically stable and is not degraded by the intestinal brush border enzymes.

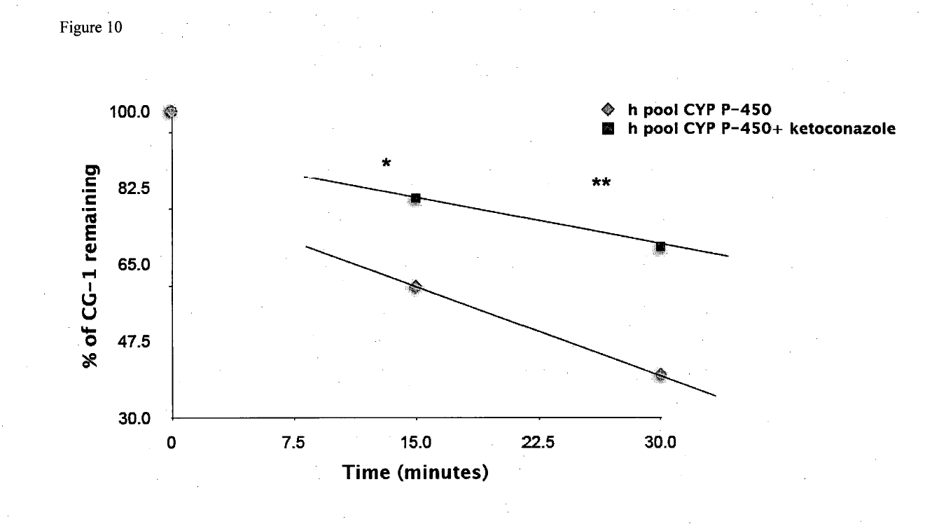

The oxidative metabolism of CG-1 was evaluated in pooled liver microsomes of rats and humans with or without 3 μM of ketoconazole (CYP3A4 inhibitor) as described above. As shown in FIG. 10 CG-1 was partially metabolized by human liver microsomes. Addition of ketoconazole (CYP3A4 inhibitor) significantly improved its stability.

### Example 12

### The Effect of Adding Ritonavir to the Pharmacokinetic Profile of CG-1

Microsomal stability and subsequent oral bioavailability of CG-1 were tested in combination with the CYP-3A4 inhibitor and protease inhibitor ritonavir.

The pharmacokinetic tests were repeated as above with the addition of ritonavir to the oral administration of CG-1. The study included three groups; the first group received 10 mg/kg of CG-1 in aqueous solution, the second one received an emulsion of 10 mg/kg of CG-1 and 10 mg/kg of ritonavir and the third group received CG-1 in the same emulsion without ritonavir.

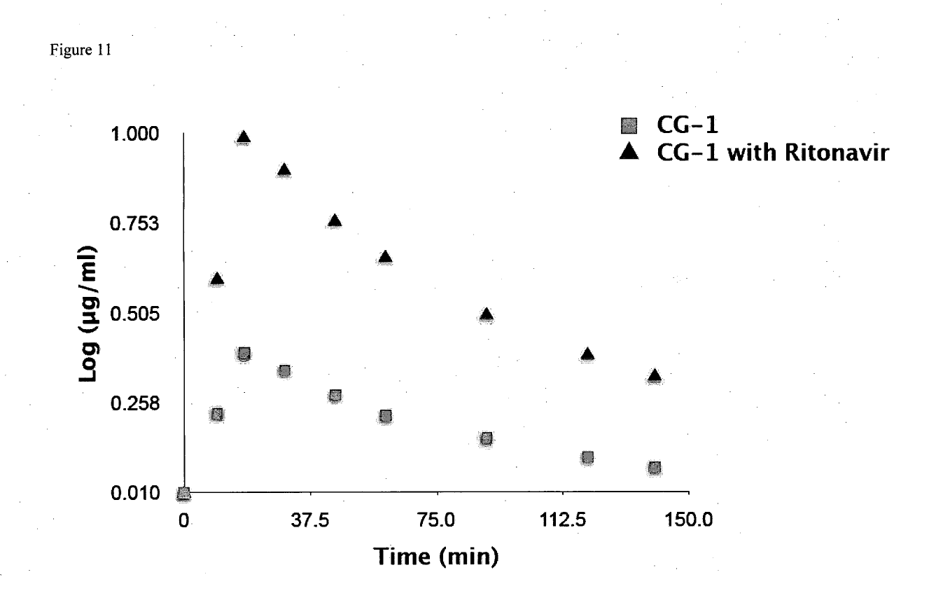

As demonstrated in FIG. 11 and Table 2, adding ritonavir improved the AUC significantly and decelerated the elimination of CG-1, therefore prolonging the elimination half life from 73±6 to 93±8 minutes. Cmax was 3 fold higher (from 866±76 to 2771±243 μg/ml) when ritonavir was added. AUC also tripled from 362±63 to 907±107 hr×μg/mL. The absolute oral bioavailability was tripled from 7.3±1.6% to 21±3%.

TABLE 2
Pharmacokinetic parameters obtained following PO administration
of CG-1 to Wistar rats with or without ritonavir (RTV) (n = 5).
Data is presented as average ± SEM. The raw data was analyzed
using non-compartmental analysis:

| PK parameters | PO | PO with RTV |
|---|---|---|
| Cmax (μg/mL) | 866 ± 76 | 2771 ± 243 |
| Tmax (min) | 18 ± 1.5 | 18 ± 2 |
| AUC (hr × μg/mL) | 362 ± 63 | 907 ± 107 |
| T_{1/2} (min) | 73 ± 6 | 93 ± 8 |
| Oral bioavailability | 7.3% ± 1.6% | 21% ± 3% |

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A macrocyclic CD4 mimetic which is a compound according to Formula II: 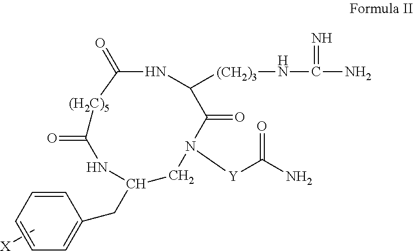
wherein X is hydrogen or is an electron withdrawing group, and Y is selected from the group consisting of: (CH₂)ₙ wherein n is 1-5; and CHR wherein R is an amino acid side chain.

2. The macrocyclic CD4 mimetic according to claim 1 wherein the electron withdrawing group is a halogen group or a hydroxyl group.

3. The macrocyclic CD4 mimetic according to claim 1 wherein X is a halogen group selected from the group consisting of: fluoride (F), chloride (Cl), bromide (Br) and iodide (I).

4. The macrocyclic CD4 mimetic according to claim 1 selected from the group consisting of: 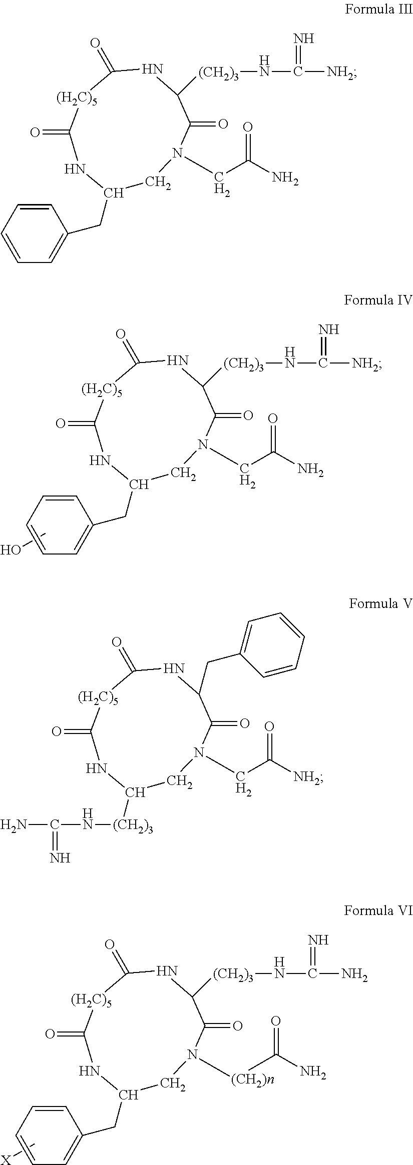
wherein X is hydrogen or is an electron withdrawing group; and n is 2-5; 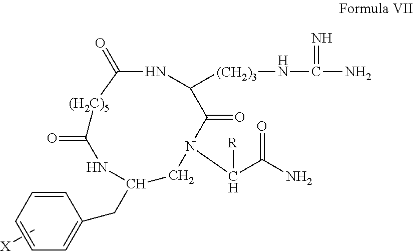
wherein X is a hydrogen or is an electron withdrawing group; and R is an amino acid side chain other than hydrogen.

5. The macrocyclic CD4 mimetic compound according to claim 1 comprising a Phe-derivative of the compound of Formula I.

6. The macrocyclic CD4 mimetic compound according to claim 5 wherein the Phe derivative is a Phe-halide derivative.

7. The macrocyclic CD4 mimetic compound according to claim 6 wherein the Phe-halide derivative is selected from the group consisting of: Phe-fluoride, Phe-chloride, Phe-bromide and Phe-iodide according to formula VIII: 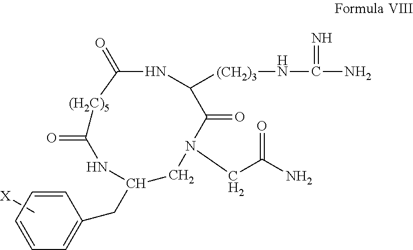
wherein X is selected from the group consisting of: fluoride (F), chloride (Cl), bromide (Br) and iodide (I).

8. The CD4 macrocyclic mimetic compound according to claim 1 comprising a urea-bond.

9. The CD4 macrocyclic mimetic compound according to claim 8 selected from Formula IX and Formula X:

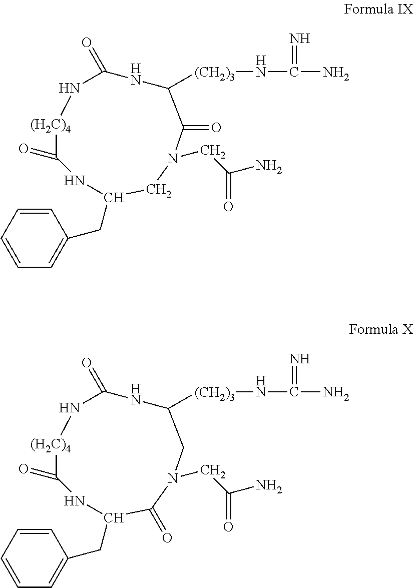

10. The CD4 macrocyclic mimetic according to claim 1 selected from the group consisting of Formula IV to Formula X, and analogs and derivatives thereof.

11. The CD4 macrocyclic mimetic according to claim 1 selected from the group consisting of Formula VI and VII.

12. The CD4 macrocyclic mimetic according to claim 1 consisting of the compound of Formula III.

13. A pharmaceutical composition comprising as an active ingredient, at least one CD4 macrocyclic mimetic compound according to claim 1, and a pharmaceutically acceptable carrier or diluent.

14. The pharmaceutical composition according to claim 13 wherein the at least one macrocyclic CD4 mimetic compound is selected from the group consisting of Formula IV to Formula X and analogs and derivatives thereof.

15. The pharmaceutical composition according to claim 13 wherein the at least one macrocyclic CD4 mimetic compound is according to Formulae III.

16. The pharmaceutical composition according to claim 13 formulated for oral administration.

17. The pharmaceutical composition according to claim 13 further comprising at least one additional retroviral inhibitor.

18. The pharmaceutical composition according to claim 17 wherein the additional retroviral inhibitor is selected from the group consisting of: 3′-azido-3′-deoxythymidine (AZT), didanosine (dideoxy inosine; ddI), zalcitabine (dideoxycytidine; ddC), tenofovir (Viread®), or lamivudine (3′-thia-2′-3′-dideoxycytidine; 3TC). Anti-retroviral compounds also include non-nucleoside reverse transcriptase inhibitors such as suramine, foscarnet-sodium, nevirapine, sustiva and tacrine; TIBO type compounds; α-APA type compounds; TAT inhibitors (e.g., RO-5-3335); protease inhibitors (e.g., indinavir, ritonavir, saquinovir); NMDA receptor inhibitors (e.g., pentamidine); α-glycosidase inhibitors (e.g., castanospermine); Rnase H inhibitors (e.g., dextran); and immunomodulating agents (e.g., levamisole, thymopentin).

19. The pharmaceutical composition according to claim 18 wherein the additional retroviral inhibitor is ritonavir.

20. The pharmaceutical composition according to claim 13 for prevention, alleviation or treatment of a viral infection.

21. A method for prevention, alleviation or treatment of a viral infection comprising administering to a subject in need thereof, a pharmaceutically active amount of a macrocyclic CD4 mimetic according to claim 1.

22. The method according to claim 21 wherein the viral infection is an HIV infection.

23. The method according to claim 21 wherein the administration is orally.

24. The method according to claim 21 wherein the administration route is selected from the group consisting of: orally, topically, intranasally, subcutaneously, intramuscularly, intravenously, intra-arterially, intraarticulary, intralesionally or parenterally.

25.-29. (canceled)
